(19) United States
(12) Patent Application Publication
(10) Pub. No.: US 2013/0316468 A1
(43) Pub. Date: Nov. 28, 2013

(54) Biomarkers for Alzheimer's Disease

(71) Applicant: Singulex, Inc., (US)

(72) Inventors: John Allan Todd, Lafayette, CA (US); Quynh Anh Thuc Lu, Mountain View, CA (US)

(21) Appl. No.: 13/833,566

(22) Filed: Mar. 15, 2013

Related U.S. Application Data
(60) Provisional application No. 61/650,246, filed on May 22, 2012.
(60) Provisional application No. 61/650,271, filed on May 22, 2012.

(51) Int. Cl. G01N 33/68

(52) U.S. Cl. CPC G01N 33/6896
USPC 436/501

(57) ABSTRACT

The disclosure is directed to the detection, early diagnosis, determination of the severity, and treatment of Alzheimer's disease (AD). A number of biomarkers and combination of biomarkers are disclosed for the determination of AD, including sTNFR2 and Abeta-42; TNFR2 and Ptau-181; sTNFR2, Abeta-42 and PTau-181; and IL-2 and Abeta oligomers. Method of treatment of AD are also disclosed.

### CROSS-REFERENCE TO RELATED APPLICATION

This application claims the benefit of U.S. provisional patent application Ser. No. 61/650,246, filed May 22, 2012, and U.S. provisional patent application Ser. No. 61/650,271, filed May 22, 2012, each of which is incorporated by reference herein in its entirety.

### FIELD

The invention relates to the detection, early diagnosis, determination of the severity, and treatment of Alzheimer's disease (AD).

### BACKGROUND

Alzheimer's disease is a chronic progressive neurodegenerative disease and is the most common form of dementia, affecting approximately 27 million people worldwide. Its cause and progression are not well understood, but it worsens overtime and eventually leads to death. Alzheimer's develops differently, with some common symptoms, making diagnosis difficult, but diagnosis is usually confirmed by behavioral and mental exams, as well as neuroimaging. More sensitive and specific detection methods need to be developed in order to prevent years of undiagnosed progression.

There is no cure for Alzheimer's disease and currently available therapeutics minimize some of the symptoms associated with AD but do not slow disease progression. Numerous experimental approaches focus on minimizing Aβ-42 levels by preventing production of or lowering Aβ-42 concentrations, stimulating the immune system to attack Aβ proteins as well as preventing Aβ proteins from aggregating and forming plaques. An important component in designing therapeutic trials is to identify patients that are at risk for developing AD such that studies can be performed in a cost effective timely manner. Beta-amyloid(1-42) (Abeta42), total tau and phospho-tau-181(pTau-181) from cerebrospinal fluid are the known biological markers to diagnose Alzheimer's disease and can be used to differentiate it from other forms of dementia. In addition, additional biomarkers would be invaluable for both understanding Aβ levels as surrogate endpoints as well as in efficient study design.

The inventors have identified a need in the art for additional biomarkers to enable more sensitive and specific detection and diagnosis of AD, including the, risk, onset and severity of AD.

### SUMMARY

In one aspect, in invention is directed to a method of determining Alzheimer's Disease in a patient. The method includes determining the concentrations of sTNFR2 and Abeta42 in a patient sample; comparing the combined concentrations of sTNFR2 and Abeta42 in the sample to the combined concentrations of sTNFR2 and Abeta42 in a healthy population, and determining Alzheimer's disease when the combined concentration of sTNRF2 and Abeta42 in the patient sample is greater than the combined concentration in a healthy population.

In another aspect, the invention is directed to a method of determining Alzheimer's disease in a patient. The method includes determining the concentrations of sTNFR2 and Ptau-181 in a patient sample; comparing the combined concentration of sTNFR2 and Ptau-181 in the sample to the combined concentration of sTNFR2 and Ptau-181 in a healthy population, and determining Alzheimer's disease when the combined concentration of sTNRF2 and Ptau-81 in the patient sample is greater than the combined concentration in a healthy population.

In a further aspect, the invention is directed to determining Alzheimer's disease in a subject. The method includes contacting, in vitro, a portion of a sample from the subject with a first antibody immunoreactive for Abeta42; contacting, in vitro, a portion of the sample from the subject with a second antibody immunoreactive for sTNFR2; contacting, in vitro, a portion of the sample from the subject with a third antibody immunoreactive for pTau-181; and determining the amounts of the Abeta42, sTNFR2 and pTau-181. The likelihood, presence or severity of Alzheimer's disease is determined by comparing, in combination, the amounts of the Abeta42, sTNFR2 and pTau-181, with amounts, in combination, Abeta42, sTNFR2 and pTau-181, in a normal health population. In various embodiments, the amount of Abeta42 in the normal healthy population is less than about 102 pg/ml, the amount of sTNFR2 in the healthy population is greater than about 748 pg/ml and/or the amount of Ptau-181 in the normal healthy population is greater than about 1.8 units/ml.

In yet another embodiment, the invention is directed to a method for diagnosing Alzheimer's Disease severity in a subject. The method includes contacting, in vitro, a portion of a sample from the subject with an antibody specific for Abeta; contacting, in vitro, a portion of the sample from the subject with an antibody immunoreactive for IL-2; determining the amounts of Abeta oligomers and IL-2 in the sample; and providing a diagnosis of the severity of Alzheimer's disease based upon the amount of Abeta and IL-2 in the patient sample. In various embodiment, the amount of Abeta oligomers is determined with a monoclonal antibody specific for Abeta42. Also, IL-2 may be detected in sub-picomolar quantities.

Still further, the invention is directed to a method of treating Alzheimer's Disease. The method includes diagnosing, prognosing or determining the severity of Alzheimer's disease by using a combination of at least two biomarkers selected from Abeta-42, Abeta oligomer, sTNFR2, PTau-181 and IL-2, and treating the patient based upon the diagnosis, prognosis or severity of the Alzheimer's disease. The combination of biomarkers may be selected from (a) sTNFR2 and Abeta-42; (b) sTNFR2 and Ptau-181; (c) sTNFR2, Abeta-42 and PTau-181; and (d) IL-2 and Abeta oligomers.

In various aspects of the invention, the sample is cerebral spinal fluid (CSF). The healthy population may include or exclude patients suffering from Parkinson's Disease. Also, the combined concentrations are determined using an area under curve analysis.

### BRIEF DESCRIPTION OF THE FIGURES

FIG. 1 shows the results of the determination of a number of markers in cerebrospinal fluid from healthy control subjects, and subjects suffering from Alzheimer's disease and Parkinson's disease.

FIG. 2 shows the results of the analysis of Abeta-42 and pTau-181 in cerebral spinal fluid from healthy control subjects, and subjects suffering from Alzheimer's disease and Parkinson's disease.

FIG. 3 shows area under curve analysis for the determination of Alzheimer's disease with a combination of Abeta-42 and pTau-181 and with a combination of Abeta-42, pTau-181 and sTNFR2.

FIG. 4 shows area under curve analysis for the determination of Alzheimer's disease with a combination of Abeta-42 and pTau-181; with a combination of Abeta-42 and sTNFR2; and with a combination of pTau-181 and sTNFR2.

FIG. 5 shows area under curve analysis for the determination of Alzheimer's disease with a combination of Abeta-42 and pTau-181 and with a combination of Abeta-42, pTau-181 and sTNFR2.

FIG. 6 shows area under curve analysis for the determination of Alzheimer's disease with a combination of Abeta-42 and pTau-181; with a combination of Abeta-42 and sTNFR2; and with a combination of pTau-181 and sTNFR2.

FIG. 7 shows scatter plots of single biomarkers for the determination of Alzheimer's disease versus controls for pTau-181, Abeta-42 or sTNFR2.

FIG. 8 shows scatter plots of two biomarkers for the determination of Alzheimer's disease versus controls for Abeta-42 and pTau-181; Abeta-42 and sTNFR2; or pTau-181 and sTNFR2.

FIG. 9 shows a graph demonstrating that the adjusted Pearson r² (which is closely related to correlation) improves in the prediction of MMSE as biomarkers are sequentially added to the algorithm: IL-2, AB-oligomers, Abeta-42, Abeta-40, IL-22, tMMP9, GCSF, pTau181, sTNFR2, and MMP2.

FIG. 10 shows a graph demonstrating a correlation between MMSE and Predicted MMSE by biomarkers IL-2 and AB-oligomers. The Spearman rank correlation of r=0.78 was calculated using SAS 9.2 with a general linear model (4.10-3.13 log₁₀ (IL-2)+1.83 log₁₀ (AB-oligomers)) and biomarkers added by forward selection.

FIG. 11 shows the results of the analysis of sTNFR2 and IFNgamma in cerebral spinal fluid from healthy control subjects, and subjects suffering from Alzheimer's disease and Parkinson's disease. For IFNgamma, all the controls were detectable, but 6/16 AD samples and 1/5 PD samples were undetectable.

FIG. 12 shows the results of the analysis of IL-6 and M-CSF in cerebral spinal fluid from healthy control subjects, and subjects suffering from Alzheimer's disease and Parkinson's disease. IL-6 was unexpectedly elevated in PD patients but not AD patients. There was some evidence of elevation of M-CSF in AD patients and a decrease in PD patients.

FIG. 13 shows the results of the analysis of hVEGF and CRP in cerebral spinal fluid from healthy control subjects, and subjects suffering from Alzheimer's disease and Parkinson's disease. There was some evidence of elevation of hVEGF in AD patients.

FIG. 14 shows the results of the analysis of TNF-alpha and IL-1b in cerebral spinal fluid from healthy control subjects, and subjects suffering from Alzheimer's disease and Parkinson's disease. For IL-1b, 5/10 controls were undetectable, 12/16 AD were undetectable and 5/5 PD were undetectable.

FIG. 15 shows the results of the analysis of GSKb and IL-1a in cerebral spinal fluid from healthy control subjects, and subjects suffering from Alzheimer's disease and Parkinson's disease. For IL-1a, 3/10 controls were undetectable, 4/16 AD were undetectable and 3/5 PD were undetectable.

FIG. 16 shows the results of the analysis of IL-8 and IL-17a in cerebral spinal fluid from healthy control subjects, and subjects suffering from Alzheimer's disease and Parkinson's disease.

FIG. 17 shows the results of the analysis of IL-13 and IL-17AF in cerebral spinal fluid from healthy control subjects, and subjects suffering from Alzheimer's disease and Parkinson's disease. For IL-13, 3/10 controls were undetectable, 9/16 AD were undetectable and 2/5 PD were undetectable.

FIG. 18 shows the results of the analysis of MIP1a and IL-10 in cerebral spinal fluid from healthy control subjects, and subjects suffering from Alzheimer's disease and Parkinson's disease.

FIG. 19 shows the results of the analysis of IL-12 and IL-2 in cerebral spinal fluid from healthy control subjects, and subjects suffering from Alzheimer's disease and Parkinson's disease. For IL-12, 7/10 controls were undetectable, 12/16 AD were undetectable and 4/5 PD were undetectable.

FIG. 20 shows the results of the analysis of IL-5 (DE's only) and sTNR1 in cerebral spinal fluid from healthy control subjects, and subjects suffering from Alzheimer's disease and Parkinson's disease.

FIG. 21 shows the results of the analysis of Abeta-40 and IL-11 in cerebral spinal fluid from healthy control subjects, and subjects suffering from Alzheimer's disease and Parkinson's disease.

FIG. 22 shows the results of the analysis of IL-21 (DE's only) and IL-17F in cerebral spinal fluid from healthy control subjects, and subjects suffering from Alzheimer's disease and Parkinson's disease.

FIG. 23 shows the results of the analysis of GCSF and GMCSF in cerebral spinal fluid from healthy control subjects, and subjects suffering from Alzheimer's disease and Parkinson's disease.

FIG. 24 shows the results of the analysis of IL-1RA and AB-oligomers in cerebral spinal fluid from healthy control subjects, and subjects suffering from Alzheimer's disease and Parkinson's disease.

FIG. 25 shows the results of the analysis of IL-7 and IL-22 in cerebral spinal fluid from healthy control subjects, and subjects suffering from Alzheimer's disease and Parkinson's disease.

FIG. 26 shows the results of the analysis of MMP2 and IL-4 in cerebral spinal fluid from healthy control subjects, and subjects suffering from Alzheimer's disease and Parkinson's disease.

FIG. 27 shows the results of the analysis of IL-15 and tMMP9 in cerebral spinal fluid from healthy control subjects, and subjects suffering from Alzheimer's disease and Parkinson's disease.

### DESCRIPTION

The invention is directed to the determination of the risk, onset, progression or severity of Alzheimer's disease. The invention also relates to the treatment of Alzheimer's disease.

As used herein, the singular foams “a,” “an,” and “the” include plural referents unless the context clearly dictates otherwise.

As used herein, the term “subject” refers to a human diseased patient or a member of a healthy population. The terms “subject” and “patient” are used herein interchangeably in many cases.

As used herein, the team “healthy population” or “control subject(s),” refers to the population of, or individual control subjects, that do not have Alzheimer's disease. In some instances the healthy population or control subjects may have Parkinson's disease as described more fully herein.

As used herein, the term “therapy” refers to the administration of any medical treatment (e.g., pharmaceuticals) or interventional treatment (e.g., surgery) to treat Alzheimer's disease.

As used herein, the terms “combined concentration(s)” or “a value representing the combination of the concentration(s)” of various biomarkers refers to a value that can be calculated from measured concentrations of the biomarkers in a sample. In its simplest form, the value may be the sum of the concentrations. In other embodiments, the measured concentrations may be weighted or used in a statistical analysis as known to those of skill in the art, for example an area under curve analysis.

In one aspect, the invention is directed to a method of determining Alzheimer's disease in a patient. The method includes determining the concentrations of soluble Tumor Necrosis Factor Receptor 2 (sTNFR2) and Amyloid beta protein 42 (Abeta-42 or Aβ-42) in a patient sample. Amyloid beta proteins (40 and 42 amino acids, Aβ-40 and Aβ-42) are the main constituent of amyloid plaques in the brains of Alzheimer's disease (AD) patients. In healthy and diseased states Aβ-40 is the more common form (10-20× higher than Aβ-42) of the two in both cerebrospinal fluid (CSF) and plasma. In patients with AD, Aβ-42 primarily aggregates and deposits in the brain forming plaques. Thus the concentration of Aβ-42 is decreased in the CSF of many AD patients. Recent studies suggest that a decrease in Aβ-42 concentrations (with a paralleled change in the ratio of Aβ-40/Aβ-42) in CSF and plasma are predictive of the onset of AD.

TNFR2 (also known as: TNFBR; TNFR75; p75; TBPII; TNFRSF1B; CD120b; TNFR1B; TNFR80; TNF-R75; p75TNFR; TNF-R-II) is the larger of the two tumor necrosis factor (TNF) receptors. It is present on many cell types, especially those of myeloid origin, and is strongly expressed on stimulated T and B lymphocytes. Increased levels of soluble TNFR2 (sTNFR2) are observed in acute and chronic neuroinflammation as well as in number of neurodegenerative conditions including ischemic stroke, Alzheimer's (AD), Parkinson's (PD), amyotrophic lateral sclerosis (ALS), and multiple sclerosis (MS); making sTNFR2 an attractive target for diagnosing neurodegenerative conditions, such as Alzheimer's Disease.

In accordance with the invention, a value representing the combined concentrations of sTNFR2 and Abeta42 in the sample are compared to a value reflecting the combined concentration of sTNFR2 and Abeta42 in a healthy population. Alzheimer's disease can be determined, for example, when the combined concentration of sTNRF2 and Abeta42 in the patient sample is greater than the combined concentration in a healthy population.

It was noted that in the brains of patients with Alzheimer's disease, the neuronal cytoskeleton is progressively disrupted and replaced by tangles of paired helical filaments, and that these filaments are composed mainly of hyperphosphorylated forms of tau. Of a potential 79 serine and threonine phosphorylation sites in the longest isoform of tau, 39 different sites have been verified. It is not clear if tau phosphorylation plays a critical role in regulating the susceptibility of the protein to aggregate, however, studies suggest that tau pathology may be downstream of the amyloidogenic cascade in AD, but it is clear that tau pathology alone causes neurodegeneration as exemplified by familial and sporadic tauopathies. Tau phosphorylated at threonine 181 (Ptau-181) has been shown to be useful for discriminating Alzheimer's disease from non-AD dementias in autopsy-confirmed dementia patients.

Accordingly, in another embodiment, the invention is directed to method of determining Alzheimer's disease in a patient by determining a value representing the concentrations of sTNFR2 and Ptau-181 in a patient sample. The method includes comparing the combined concentration of sTNFR2 and Ptau-181 in the sample to a value reflecting the combined concentration of sTNFR2 and Ptau-181 in a healthy population. Alzheimer's disease is determined, for example, when the combined concentrations of sTNRF2 and Ptau-81 in the patient sample is greater than the combined concentration in a healthy population.

In another embodiment, the invention is directed to determining Alzheimer's disease in a subject by determining a value representing the concentrations of a combination of sTNFR2, Ptau-181 and Abeta-42 in a patient sample and comparing the value to a value reflecting the combined concentration of sTNFR2, Ptau-181 Abeta-42 in a healthy population. Alzheimer's disease is determined, for example, when the combined concentrations of sTNRF2, Ptau-81 and Abeta-42 in the patient sample is greater than the combined concentration in a healthy population.

In another aspect, the invention is directed to a method for determining Alzheimer's disease in a subject. The method includes contacting, in vitro, a portion of a sample from the subject with a first antibody immunoreactive for Abeta42; contacting, in vitro, a portion of the sample from the subject with a second antibody immunoreactive for sTNFR2; contacting, in vitro, a portion of the sample from the subject with a third antibody immunoreactive for pTau-181. The amounts of the Abeta42, sTNFR2 and pTau-181 are determined. The likelihood, presence or severity of Alzheimer's disease is determined by comparing, in combination, the amounts of the Abeta42, sTNFR2 and pTau-181, or a value representing the combined amounts, with amounts, in combination, or a value representing the combined amounts, of Abeta42, sTNFR2 and pTau-181, in a normal health population. In various aspects of the method, the amount of Abeta42 in the normal healthy population is less than about 102 pg/ml, the amount of sTNFR2 in the healthy population is greater than about 748 pg/ml, and/or, the amount of Ptau-181 in the normal healthy population is greater than about 1.8 units/ml.

In yet another embodiment, the invention is directed to a method for diagnosing Alzheimer's Disease severity (MMSE score) in a subject. The mini-mental state examination (MMSE) or Folstein test is a brief 30-point questionnaire test that can be used to screen for cognitive impairment, and was used in this study to severity in AD patients. The method includes contacting, in vitro, a portion of a sample from the subject with an antibody specific for Abeta; contacting, in vitro, a portion of the sample from the subject with an antibody immunoreactive for IL-2; determining the amounts of Abeta oligomers and IL-2 in the sample; and providing a diagnosis of the severity of Alzheimer's disease based upon the amount of Abeta and IL-2 in the patient sample. In various aspects of the method, the amount of Abeta oligomers is determined with a monoclonal antibody specific for Abeta42, the sample is CSF, and Abeta oligomers and IL-2 are detected in sub picomolar quantities.

Interleukin-2 (IL2), is an immunoregulatory lymphokine that is produced by antigen-activated T-cells. It is produced not only by mature T lymphocytes on stimulation, but also constitutively by certain T-cell lymphoma cell lines. It is useful in the study of the molecular nature of T-cell differentiation and, like interferons, augments natural killer cell activity.

Abeta oligomers are predictive of Alzheimer's Disease. Amyloid beta proteins (40 and 42 amino acids) are the main constituent of amyloid plaques in the brains of Alzheimer's disease (AD) patients. In healthy and diseased states Aβ-40 is the more common form (10-20× higher than Aβ-42) of the two in both cerebrospinal fluid (CSF) and plasma. In patients with AD, Aβ-42 primarily aggregates and deposits in the brain forming plaques. Thus the concentration of Aβ-42 is decreased in the CSF of many AD patients. Studies suggest that a decrease in Aβ-42 concentrations (with a paralleled change in the ratio of Aβ-40/Aβ-42) in CSF and plasma are predictive of the onset of AD. However, the quantity of amyloid β-protein containing plaques does not correlate well with clinical status and data suggest that soluble, non-plaque oligomers of amyloid beta proteins are more strongly associated with AD.

Preventive therapy is a major focus as the best way to manage AD. Current FDA-approved Alzheimer's drugs operate through two different mechanisms. Cholinesterase inhibitors work by slowing down the disease activity that breaks down a key neurotransmitter. These include donepezil (ARICEPT®), galantamine (RAZADYNE®), rivastigmine (NAMENDA®) and tacrine (EXELON®). Memantine (COGNEX®), the fifth Alzheimer's drug, is an NMDA (N-methyl-D-aspartate) receptor antagonist, which works by regulating the activity of glutamate, a chemical messenger involved in learning and memory. Memantine protects brain cells against excess glutamate, a chemical messenger released in large amounts by cells damaged by Alzheimer's disease and other neurological disorders. Attachment of glutamate to cell surface “docking sites” called NMDA receptors permits calcium to flow freely into the cell. Over time, this leads to chronic overexposure to calcium, which can speed up cell damage. Memantine prevents this destructive chain of events by partially blocking the NMDA receptors. On average, the five approved Alzheimer's drugs are effective for about six to 12 months for about half of the individuals who take them.

Accordingly, in one aspect, the invention is directed to a method of treating Alzheimer's disease. The method includes diagnosing, prognosing or determining the severity of Alzheimer's disease in accordance with the methods described herein. Researchers are looking for new ways to treat Alzheimer's. Current drugs help mask the symptoms of Alzheimer's, but do not treat the underlying disease. A breakthrough Alzheimer's drug would treat the underlying disease and stop or delay the cell damage that eventually leads to the worsening of symptoms. There are several promising drugs in development and testing, and the treatment of the invention includes the use of these therapies available in the future.

The diagnosing, prognosing or determining the severity of Alzheimer's disease can be accomplished by using a combination of at least two biomarkers selected from Abeta-42, Abeta oligomer, sTNFR2, PTau-181 and IL-2 as described herein. For example, the combination of biomarkers can be one of the following: (1) sTNFR2 and Abeta-42; (2) sTNFR2 and Ptau-181; (4) sTNFR2, Abeta-42 and PTau-181; and (4) IL-2 and Abeta oligomers.

Guidelines describe the need for non-invasive biomarkers that can be used to predict and diagnose the formation of AD. Such information will be invaluable for clinical study design, as well as the evaluation of therapeutic effectiveness. Measuring Aβ-40 and Aβ-42 concentrations in plasma provide promise for such information. In healthy normal humans, plasma concentrations range from 200-400 pg/ml (Aβ-40) and 15-30 pg/ml (Aβ-42). However with AD, Aβ-42 levels decrease, and are often undetectable by currently available EIA technology. Furthermore, interventional strategies based on depleting Aβ-42 formation require methods that measure decreases in Aβ-42. Thus there is a need to accurately and precisely quantify low concentrations of amyloid proteins in plasma.

Use of the Aβ-40 and Aβ-42 assays in conjunction with other with exceptional sensitivity, enabling the use of Aβ-40/Aβ-42 as a velocity biomarker in Alzheimer's disease studies and to evaluate therapeutic interventions. Among other advantages, this assay allows investigators to: (1) identify subjects with potential high risk for developing AD and hence design interventional studies that include high risk for disease development; (2) design more robust clinical and preclinical studies when Aβ protein concentrations are used as a therapeutic endpoint; and (3) understand how Aβ protein levels change in humans as they transition from a healthy to a diseased state.

The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. It will be appreciated that the choice of epitope or region of the molecule to which the antibody is raised will determine its specificity, e.g., for various fauns of the molecule, if present, or for total (e.g., all, or substantially all of the molecule). In some embodiments, the antibody is a polyclonal antibody. In other embodiments, the antibody is a monoclonal antibody.

Particular antibodies useful for the present invention include an anti-pTau-181 antibody from Theinio Scientific. Antibodies specific for Abeta-42 can be obtained from Covance, Inc. (Princeton, N.J.). sTNFR2 and IL-2 specific antibodies can be obtained from R&D Systems, Inc. Other antibodies useful for the invention are available from numerous commercial sources.

Those of skill in the art will appreciate that statistical approaches have been developed to combine the data from multiple marker and provide a statistical likelihood of the presence of a biological state, e.g., the presence of a disease such as cancer. Examples of such methods are disclosed in U.S. patent application Ser. Nos. 11/934,008 (U.S. Pat. No. 7,998,743); 11/939,484 (US 2008/0254481); and 11/640,511 (US 2007/0161062), each of which is incorporated herein in their entireties. In one embodiment, the concentration of the panel members in a patient sample can be combined using a logistical regression and the disease status of the subject can be determined using a Receiver-Operating Characteristic (ROC) analysis. See, e.g., U.S. patent application Ser. Nos. 11/934,008 (U.S. Pat. No. 7,998,743); 11/939,484 (US 2008/0254481); and 11/640,511 (US 2007/0161062). In other approaches, statistical methods can be used to classify the sample based on the detection of the marker panels. For example, the results of the marker assays can be used to classify a sample as diseased or healthy. Such classification (pattern recognition) methods include, e.g., Bayesian classifiers, profile similarity, artificial neural networks, support vector machines (SVM), logistic or logic regression, linear or quadratic discriminant analysis, decision trees, clustering, principal component analysis, Fischer's discriminate analysis or nearest neighbor classifier analysis. Machine learning approaches to classification include, e.g., weighted voting, k-nearest neighbors, decision tree induction, support vector machines (SVM), and feed-forward neural networks. Such methods are known to those of skill in the art.

In various aspects of the invention, the markers are identified in a single molecule detection system for the highly sensitive detection of the markers. In one aspect, the invention provides a method for determining the presence or absence of a single molecule, e.g., a molecule of a marker of a biological state, in a sample, by i) labeling the molecule if present, with a label; and ii) detecting the presence or absence of the label, where the detection of the presence of the label indicates the presence of the single molecule in the sample. In some embodiments, the method is capable of detecting the molecule at a limit of detection of less than about 100, 80, 60, 50, 40, 30, 20, 15, 12, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001 femtomolar. Detection limits may be determined by use of an appropriate standard, e.g., National Institute of Standards and Technology reference standard material. “Limit of detection,” or LoD, as those terms are used herein, includes the lowest concentration at which one can identify a sample as containing a molecule of the substance of interest, e.g., the first non-zero value. It can be defined by the variability of zeros and the slope of the standard curve. For example, the limit of detection of an assay may be determined by running a standard curve, determining the standard curve zero value, and adding 2 standard deviations to that value. A concentration of the substance of interest that produces a signal equal to this value is the “lower limit of detection” concentration.

In some embodiments, the methods of the present invention are capable of detecting the Aβ-40 at a limit of detection of less than about 100, 80, 60, 50, 30, 20, 10, 5, 1, 0.5, 0.1, 0.05, 0.01, 0.005, 0.001, 0.0005 or 0.0001 pg/ml, e.g., less than about 100 pg/ml. In some embodiments, the method is capable of detecting Aβ-40 at a limit of detection of less than about 100 pg/ml. In some embodiments, the method is capable of detecting the Aβ-40 a limit of detection of less than about 80 pg/ml. In some embodiments, the method is capable of detecting the Aβ-40 a limit of detection of less than about 60 pg/ml. In some embodiments, the method is capable of detecting the Aβ-40 a limit of detection of less than about 50 pg/ml. In some embodiments, the method is capable of detecting the Aβ-40 a limit of detection of less than about 30 pg/ml. In some embodiments, the method is capable of detecting the Aβ-40 a limit of detection of less than about 25 pg/ml. In some embodiments, the method is capable of detecting the Aβ-40 a limit of detection of less than about 10 pg/ml. In some embodiments, the method is capable of detecting the Aβ-40 a limit of detection of less than about 5 pg/ml. In some embodiments, the method is capable of detecting the Aβ-40 a limit of detection of less than about 1 pg/ml. In some embodiments, the method is capable of detecting the Aβ-40 a limit of detection of less than about 0.5 pg/ml. In some embodiments, the method is capable of detecting the Aβ-40 at a limit of detection of less than about 0.1 pg/ml. In some embodiments, the method is capable of detecting the Aβ-40 at a limit of detection of less than about 0.05 pg/ml. In some embodiments, the method is capable of detecting the Aβ-40 at a limit of detection of less than about 0.01 pg/ml. In some embodiments, the method is capable of detecting the Aβ-40 at a limit of detection of less than about 0.005 pg/ml. In some embodiments, the method is capable of detecting the Aβ-40 at a limit of detection of less than about 0.001 pg/ml. In some embodiments, the method is capable of detecting the Aβ-40 at a limit of detection of less than about 0.0005 pg/ml. In some embodiments, the method is capable of detecting the Aβ-40 at a limit of detection of less than about 0.0001 pg/ml.

In some embodiments, the method is capable of detecting the Aβ-42 at a limit of detection of less than about 250, 200, 150, 100, 80, 60, 50, 30, 20, 10, 5, 1, 0.5, 0.1, 0.05, 0.01, 0.005, 0.001, 0.0005 or 0.0001 pg/ml, e.g., less than about 200 pg/ml. In some embodiments, the method is capable of detecting Aβ-42 at a limit of detection of less than about 200 pg/ml. In some embodiments, the method is capable of detecting Aβ-42 at a limit of detection of less than about 150 pg/ml. In some embodiments, the method is capable of detecting Aβ-42 at a limit of detection of less than about 100 pg/ml. In some embodiments, the method is capable of detecting the Aβ-42 a limit of detection of less than about 80 pg/ml. In some embodiments, the method is capable of detecting the Aβ-42 a limit of detection of less than about 60 pg/ml. In some embodiments, the method is capable of detecting the Aβ-42 a limit of detection of less than about 50 pg/ml. In some embodiments, the method is capable of detecting the Aβ-42 a limit of detection of less than about 30 pg/ml. In some embodiments, the method is capable of detecting the Aβ-42 a limit of detection of less than about 25 pg/ml. In some embodiments, the method is capable of detecting the Aβ-42 a limit of detection of less than about 10 pg/ml. In some embodiments, the method is capable of detecting the Aβ-42 a limit of detection of less than about 5 pg/ml. In some embodiments, the method is capable of detecting the Aβ-42 a limit of detection of less than about 1 pg/ml. In some embodiments, the method is capable of detecting the Aβ-42 a limit of detection of less than about 0.5 pg/ml. In some embodiments, the method is capable of detecting the Aβ-42 at a limit of detection of less than about 0.1 pg/ml. In some embodiments, the method is capable of detecting the Aβ-42 at a limit of detection of less than about 0.05 pg/ml. In some embodiments, the method is capable of detecting the Aβ-42 at a limit of detection of less than about 0.01 pg/ml. In some embodiments, the method is capable of detecting the 4-42 at a limit of detection of less than about 0.005 pg/ml. In some embodiments, the method is capable of detecting the Aβ-42 at a limit of detection of less than about 0.001 pg/ml. In some embodiments, the method is capable of detecting the Aβ-42 at a limit of detection of less than about 0.0005 pg/ml. In some embodiments, the method is capable of detecting the Aβ-42 at a limit of detection of less than about 0.0001 pg/ml.

Single molecule detectors and the detection of various analytes have been described, for example, in U.S. Pat. No. 7,572,640, U.S. Patent Application Publication No. 2010/0112727, U.S. Patent Application Publication No. 2009/0159812, U.S. Pat. No. 7,838,250, and U.S. Patent Application Publication No. 2009/0234202, each of which is incorporated by reference herein in its entirety. The methods and apparatus are useful for determining a concentration of a molecule, e.g., a marker indicative of a biological state, in a sample by detecting single molecules of the molecule in the sample. The “detecting” of a single molecule includes detecting the molecule directly or indirectly. In the case of indirect detection, labels that correspond to single molecules, e.g., labels attached to the single molecules, can be detected.

In some embodiments, detecting the presence or absence of said label includes: (i) passing a portion of said sample through an interrogation space; (ii) subjecting said interrogation space to exposure to electromagnetic radiation, said electromagnetic radiation being sufficient to stimulate said fluorescent moiety to emit photons, if said label is present; and (iii) detecting photons emitted during said exposure of step (ii). The method may further include deteimining a background photon level in said interrogation space, wherein said background level represents the average photon emission of the interrogation space when it is subjected to electromagnetic radiation in the same manner as in step (ii), but without label in the interrogation space. The method may further include comparing the amount of photons detected in step (iii) to a threshold photon level, wherein said threshold photon level is a function of said background photon level, wherein an amount of photons detected in step (iii) greater that the threshold level indicates the presence of said label, and an amount of photons detected in step (iii) equal to or less than the threshold level indicates the absence of said label.

One skilled in the art will recognize that many strategies can be used for labeling target molecules to enable their detection or discrimination in a mixture of molecules. The labels may be attached by any known means, including methods that utilize non-specific or specific interactions of label and target. Labels may provide a detectable signal or affect the mobility of the target molecule in an electric field. In addition, labeling can be accomplished directly or through binding partners.

Any suitable binding partner with the requisite specificity for the form of target molecule to be detected may be used. If the molecule, e.g., a marker, has several different foams, various specificities of binding partners are possible. Suitable binding partners are known in the art and include antibodies, aptamers, lectins, and receptors. A useful and versatile type of binding partner is an antibody.

In some embodiments, the binding partner is an antibody specific for a molecule to be detected. The term “antibody,” as used herein, is a broad teen and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. It will be appreciated that the choice of epitope or region of the molecule to which the antibody is raised will determine its specificity, e.g., for various forms of the molecule, if present, or for total (e.g., all, or substantially all of the molecule).

In some embodiments, the antibody is a polyclonal antibody. In other embodiments, the antibody is a monoclonal antibody. Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988). One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press; J. Immunol. 149, 3914-3920 (1992)). Monoclonal and polyclonal antibodies also commercially available from a variety of commercial sources. (e.g., R and D Systems, Minneapolis, Minn.; HyTest Ltd., Turku Finland; Abcam Inc., Cambridge, Mass.; Life Diagnostics, Inc., West Chester, Pa.; Fitzgerald Industries International, Inc., Concord, Mass.; BiosPacific, Emeryville, Calif.).

Capture binding partners and detection binding partner pairs, e.g., capture and detection antibody pairs may be used in embodiments of the invention. Thus, in some embodiments, a heterogeneous assay protocol is used in which, typically, two binding partners, e.g., two antibodies, are used. One binding partner is a capture partner, usually immobilized on a solid support, and the other binding partner is a detection binding partner, typically with a detectable label attached. Antibody pairs can be designed and prepared by methods well known in the art. Compositions of the invention include antibody pairs wherein one member of the antibody pair is a label as described herein, and the other member is a capture antibody.

In some embodiments of labels used in the invention, the binding partner, e.g., antibody, is attached to a fluorescent moiety. The fluorescence of the moiety will be sufficient to allow detection in a single molecule detector, such as the single molecule detectors described herein.

A “fluorescent moiety,” as that term is used herein, includes one or more fluorescent entities whose total fluorescence is such that the moiety may be detected in the single molecule detectors described herein. Thus, a fluorescent moiety may include a single entity (e.g., a Quantum Dot or fluorescent molecule) or a plurality of entities (e.g., a plurality of fluorescent molecules). It will be appreciated that when “moiety,” as that term is used herein, refers to a group of fluorescent entities, e.g., a plurality of fluorescent dye molecules, each individual entity may be attached to the binding partner separately or the entities may be attached together, as long as the entities as a group provide sufficient fluorescence to be detected.

Typically, the fluorescence of the moiety involves a combination of quantum efficiency and lack of photobleaching sufficient that the moiety is detectable above background levels in a single molecule detector, with the consistency necessary for the desired limit of detection, accuracy, and precision of the assay. For example, in some embodiments, the fluorescence of the fluorescent moiety is such that it allows detection and/or quantitation of a molecule, e.g., a marker, at a limit of detection of less than about 10, 9, 8, 7, 6, 5, 4, 3, 2, 1, 0.1, 0.01, 0.001, 0.00001, or 0.000001 pg/ml and with a coefficient of variation of less than about 20, 15, 14, 13, 12, 11, 10, 9, 8, 7, 6, 5, 4, 3, 2, 1%, in the instruments described herein or other instruments that provide suitable quantitative accuracy.

Furthermore, a fluorescent moiety has properties that are consistent with its use in the assay of choice. In some embodiments, the assay is an immunoassay, where the fluorescent moiety is attached to an antibody; the moiety must have properties such that it does not aggregate with other antibodies or proteins, or experiences no more aggregation than is consistent with the required accuracy and precision of the assay. In some embodiments, fluorescent moieties that are preferred are fluorescent moieties, e.g., dye molecules that have a combination of 1) high absorption coefficient; 2) high quantum yield; 3) high photostability (low photobleaching); and 4) compatibility with labeling the molecule of interest (e.g., protein) so that it may be analyzed using the analyzers and systems, for example, as described herein (e.g., does not cause precipitation of the protein of interest, or precipitation of a protein to which the moiety has been attached).

Fluorescent moieties, e.g., a single fluorescent dye molecule or a plurality of fluorescent dye molecules, that are useful in some embodiments of the invention may be defined in terms of their photon emission characteristics when stimulated by EM radiation. For example, in some embodiments, the invention utilizes a fluorescent moiety, e.g., a moiety comprising a single fluorescent dye molecule or a plurality of fluorescent dye molecules, that is capable of emitting an average of at least about 10, 20, 30, 40, 50, 75, 100, 125, 150, 175, 200, 225, 250, 275, 300, 350, 400, 500, 600, 700, 800, 900, or 1000, photons when simulated by a laser emitting light at the excitation wavelength of the moiety, where the laser is focused on a spot of not less than about 5 microns in diameter that contains the moiety, and where the total energy directed at the spot by the laser is no more than about 15 microJoules. It will be appreciated that the total energy may be achieved by many different combinations of power output of the laser and length of time of exposure of the dye moiety. For example, in order to achieve total energy of 15 microJoules, a laser of a power output of 1 mW may be used for 15 ms, 3 mW for 5 ms, and so on.

In some embodiments, the invention utilizes a fluorescent dye moiety, e.g., a single fluorescent dye molecule or a plurality of fluorescent dye molecules, that is capable of emitting an average of at least about 200 photons when simulated by a laser emitting light at the excitation wavelength of the moiety, where the laser is focused on a spot of not less than about 5 microns in diameter that contains the moiety, and wherein the total energy directed at the spot by the laser is no more than about 15 microJoules.

In some embodiments, the fluorescent moiety includes an average of at least about 1, 2, 3, 4, 5, 6, 7, 8, 9, or 10 fluorescent entities, e.g., fluorescent molecules. In some embodiments, the fluorescent moiety includes an average of no more than about 2, 3, 4, 5, 6, 7, 8, 9, 10, or 11 fluorescent entities, e.g., fluorescent molecules. In some embodiments, the fluorescent moiety includes an average of about 1 to 11, or about 2 to 10, or about 2 to 8, or about 2 to 6, or about 2 to 5, or about 2 to 4, or about 3 to 10, or about 3 to 8, or about 3 to 6, or about 3 to 5, or about 4 to 10, or about 4 to 8, or about 4 to 6, or about 2, 3, 4, 5, 6, or more than about 6 fluorescent entities. By “average” it is meant that, in a given sample that is representative of a group of labels of the invention, where the sample contains a plurality of the binding partner-fluorescent moiety units, the molar ratio of the particular fluorescent entity to the binding partner, as determined by standard analytical methods, corresponds to the number or range of numbers specified. For example, in embodiments wherein the label includes a binding partner that is an antibody and a fluorescent moiety that includes a plurality of fluorescent dye molecules of a specific absorbance, a spectrophotometric assay can be used in which a solution of the label is diluted to an appropriate level and the absorbance at 280 nm is taken to determine the molarity of the protein (antibody) and an absorbance at, e.g., 650 nm (for ALEXA FLUOR® 647), is taken to determine the molarity of the fluorescent dye molecule. The ratio of the latter molarity to the former represents the average number of fluorescent entities (dye molecules) in the fluorescent moiety attached to each antibody.

A non-exclusive list of useful fluorescent entities for use in the fluorescent moieties of the invention is given in Table 2 of U.S. Patent Publication No. 2009/0234202, which includes a number of ALEXA FLUOR® dyes, Atto dyes (Attec-tec GmbH, Germany), and Dyomic Fluors (Dyomics GmbH, Germany).

Other suitable dyes for use in the invention include modified carbocyanine dyes. One such modification includes modification of an indolium ring of the carbocyanine dye to permit a reactive group or conjugated substance at the number three position. The modification of the indolium ring provides dye conjugates that are uniformly and substantially more fluorescent on proteins, nucleic acids and other biopolymers, than conjugates labeled with structurally similar carbocyanine dyes bound through the nitrogen atom at the number one position. In addition to having more intense fluorescence emission than structurally similar dyes at virtually identical wavelengths, and decreased artifacts in their absorption spectra upon conjugation to biopolymers, the modified carbocyanine dyes have greater photostability and higher absorbance (extinction coefficients) at the wavelengths of peak absorbance than the structurally similar dyes. Thus, the modified carbocyanine dyes result in greater sensitivity in assays using the modified dyes and their conjugates. Preferred modified dyes include compounds that have at least one substituted indolium ring system in which the substituent on the 3-carbon of the indolium ring contains a chemically reactive group or a conjugated substance. Other dye compounds include compounds that incorporate an azabenzazolium ring moiety and at least one sulfonate moiety. The modified carbocyanine dyes that can be used to detect individual molecules in various embodiments of the invention are described in U.S. Pat. No. 6,977,305, which is herein incorporated by reference in its entirety. Thus, in some embodiments the labels of the invention utilize a fluorescent dye that includes a substituted indolium ring system in which the substituent on the 3-carbon of the indolium ring contains a chemically reactive group or a conjugated substance group.

The ALEXA FLUOR® dyes are disclosed in U.S. Pat. Nos. 6,977,305; 6,974,874; 6,130,101; and 6,974,305 which are herein incorporated by reference in their entirety. Some embodiments of the invention utilize a dye chosen from the group consisting of ALEXA FLUOR® 647, ALEXA FLUOR® 488, ALEXA FLUOR® 532, ALEXA FLUOR® 555, ALEXA FLUOR® 610, ALEXA FLUOR® 680, ALEXA FLUOR® 700, and ALEXA FLUOR® 750. Some embodiments of the invention utilize the ALEXA FLUOR® 647 molecule, which has an absorption maximum between about 650 and 660 nm and an emission maximum between about 660 and 670 nm. The ALEXA FLUOR® 647 dye is used alone or in combination with other ALEXA FLUOR® dyes.

Currently available organic fluors can be improved by rendering them less hydrophobic by adding hydrophilic groups such as polyethylene. Alternatively, currently sulfonated organic fluors such as the ALEXA FLUOR® 647 dye can be rendered less acidic by making them zwitterionic. Particles such as antibodies that are labeled with the modified fluors are less likely to bind non-specifically to surfaces and proteins in immunoassays, and thus enable assays that have greater sensitivity and lower backgrounds. Methods for modifying and improving the properties of fluorescent dyes for the purpose of increasing the sensitivity of a system that detects single molecules are known in the art. Preferably, the modification improves the Stokes shift while maintaining a high quantum yield.

In some embodiments, the fluorescent label moiety that is used to detect a molecule in a sample using the analyzer systems of the invention is a quantum dot. Quantum dots (QDs), also known as semiconductor nanocrystals or artificial atoms, are semiconductor crystals that contain anywhere between 100 to 1,000 electrons and range from 2-10 nm. Some QDs can be between 10-20 nm in diameter. QDs have high quantum yields, which makes them particularly useful for optical applications. QDs are fluorophores that fluoresce by forming excitons, which are similar to the excited state of traditional fluorophores, but have much longer lifetimes of up to 200 nanoseconds. This property provides QDs with low photobleaching. The energy level of QDs can be controlled by changing the size and shape of the QD, and the depth of the QDs' potential. One optical feature of small excitonic QDs is coloration, which is determined by the size of the dot. The larger the dot, the redder, or more towards the red end of the spectrum the fluorescence. The smaller the dot, the bluer or more towards the blue end it is. The bandgap energy that determines the energy and hence the color of the fluoresced light is inversely proportional to the square of the size of the QD. Larger QDs have more energy levels which are more closely spaced, thus allowing the QD to absorb photons containing less energy, i.e., those closer to the red end of the spectrum. Because the emission frequency of a dot is dependent on the bandgap, it is possible to control the output wavelength of a dot with extreme precision. In some embodiments the protein that is detected with the single molecule analyzer system is labeled with a QD. In some embodiments, the single molecule analyzer is used to detect a protein labeled with one QD and using a filter to allow for the detection of different proteins at different wavelengths.

QDs have broad excitation and narrow emission properties which, when used with color filtering, require only a single electromagnetic source to resolve individual signals during multiplex analysis of multiple targets in a single sample. Thus, in some embodiments, the analyzer system includes one continuous wave laser and particles that are each labeled with one QD. Colloidally prepared QDs are free floating and can be attached to a variety of molecules via metal coordinating functional groups. These groups include but are not limited to thiol, amine, nitrile, phosphine, phosphine oxide, phosphonic acid, carboxylic acids or other ligands. By bonding appropriate molecules to the surface, the quantum dots can be dispersed or dissolved in nearly any solvent or incorporated into a variety of inorganic and organic films. Quantum dots (QDs) can be coupled to streptavidin directly through a maleimide ester coupling reaction or to antibodies through a meleimide-thiol coupling reaction. This yields a material with a biomolecule covalently attached on the surface, which produces conjugates with high specific activity. In some embodiments, the protein that is detected with the single molecule analyzer is labeled with one quantum dot. In some embodiments, the quantum dot is between 10 and 20 nm in diameter. In other embodiments, the quantum dot is between 2 and 10 nm in diameter. In other embodiments, the quantum dot is about 2 nm, 3 nm, 4 nm, 5 nm, 6 nm, 7 nm, 8 nm, 9 nm, 10 nm, 11 nm, 12 nm, 13 nm, 14 nm, 15 v, 16 nm, 17 nm, 18 nm, 19 nm or 20 nm in diameter. Quantum Dots useful in the invention include QDOT® 525, QDOT® 565, QDOT® 585, QDOT® 605, QDOT® 655, QDOT® 705, and QDOT® 800 (Life Technologies, Inc.).

Attachment of the fluorescent moiety, or fluorescent entities that make up the fluorescent moiety, to the binding partner, e.g., antibody, may be by any suitable means; such methods are well-known in the art and exemplary methods are given in the Examples. In some embodiments, after attachment of the fluorescent moiety to the binding partner to form a label for use in the methods of the invention, and prior to the use of the label for labeling the protein of interest, it is useful to perform a filtration step. E.g., an antibody-dye label may be filtered prior to use, e.g., through a 0.2 micron filter, or any suitable filter for removing aggregates. Other reagents for use in the assays of the invention may also be filtered, e.g., through a 0.2 micron filter, or any suitable filter. Without being bound by theory, it is thought that such filtration removes a portion of the aggregates of the, e.g., antibody-dye labels. As such aggregates can bind as a unit to the protein of interest, but upon release in elution buffer are likely to disaggregate, false positives may result; i.e., several labels will be detected from an aggregate that has bound to only a single protein molecule of interest. Regardless of theory, filtration has been found to reduce false positives in the subsequent assay and to improve accuracy and precision.

It will be appreciated that immunoassays often employ a sandwich format, in which binding partner pairs, e.g., antibodies, to the same molecule, e.g., a marker, are used. The invention also encompasses binding partner pairs, e.g., antibodies, wherein both antibodies are specific to the same molecule, e.g., the same marker, and wherein at least one member of the pair is a label as described herein. Thus, for any label that includes a binding-partner and a fluorescent moiety, the invention also encompasses a pair of binding partners wherein the first binding partner, e.g., antibody, is part of the label, and the second binding painter, e.g., antibody, is, typically, unlabeled and serves as a capture binding partner. In addition, binding partner pairs are frequently used in FRET assays. FRET assays useful in the invention are disclosed in U.S. Patent Publication No. 2006/0078998, incorporated by reference herein in its entirety, and the present invention also encompasses binding partner pairs, each of which includes a FRET label.

In general, any method of sample preparation may be used that produces a label corresponding to a molecule of interest, e.g., a marker of a biological state to be measured, where the label is detectable in analytical instruments, such as for example the single molecule detector as described herein. As is known in the art, sample preparation in which a label is added to one or more molecules may be performed in a homogeneous or heterogeneous format. In some embodiments, the sample preparation is formed in a homogenous format. In analyzer systems employing a homogenous format, unbound label is not removed from the sample. See, e.g., U.S. Pat. No. 7,572,640. In some embodiments, the particle or particles of interest are labeled by addition of labeled antibody or antibodies that bind to the particle or particles of interest.

In some embodiments, a heterogeneous assay format is used, where, typically, a step is employed for removing unbound label. Such assay formats are well-known in the art. One particularly useful assay format is a sandwich assay, e.g., a sandwich immunoassay. In this format, the molecule of interest, e.g., marker of a biological state, is captured, e.g., on a solid support, using a capture binding partner. Unwanted molecules and other substances may then optionally be washed away, followed by binding of a label comprising a detection binding partner and a detectable label, e.g., fluorescent moiety. Further washes remove unbound label, then the detectable label is released, usually though not necessarily still attached to the detection binding partner. In alternative embodiments, sample and label are added to the capture binding partner without a wash in between, e.g., at the same time. Other variations will be apparent to one of skill in the art.

In some embodiments, the method for detecting the molecule of interest, e.g., marker of a biological state, uses a sandwich assay with antibodies, e.g., monoclonal antibodies as capture binding pay biers. The method includes binding molecules in a sample to a capture antibody that is immobilized on a binding surface, and binding the label comprising a detection antibody to the molecule to form a “sandwich” complex. The label includes the detection antibody and a fluorescent moiety, as described herein, which is detected, e.g., using the single molecule analyzers of the invention. Both the capture and detection antibodies specifically bind the molecule.

The capture binding partner may be attached to a solid support, e.g., a microtiter plate or paramagnetic beads. In some embodiments, the invention provides a binding pai bier for a molecule of interest, e.g., marker of a biological state, attached to a paramagnetic bead. Any suitable binding partner that is specific for the molecule that it is wished to capture may be used. The binding partner may be an antibody, e.g., a monoclonal antibody. It will be appreciated that antibodies identified herein as useful as a capture antibody may also be useful as detection antibodies, and vice versa.

The attachment of the binding partner, e.g., antibody, to the solid support may be covalent or noncovalent. In some embodiments, the attachment is noncovalent. An example of a noncovalent attachment well-known in the art is biotin-avidin/streptavidin interactions. Thus, in some embodiments, a solid support, e.g., a microtiter plate or a paramagnetic bead, is attached to the capture binding partner, e.g., antibody, through noncovalent attachment, e.g., biotin-avidin/streptavidin interactions. In some embodiments, the attachment is covalent. Thus, in some embodiments, a solid support, e.g., a microtiter plate or a paramagnetic bead, is attached to the capture binding partner, e.g., antibody, through covalent attachment.

The capture antibody can be covalently attached in an orientation that optimizes the capture of the molecule of interest. For example, in some embodiments, a binding partner, e.g., an antibody, is attached in a orientated manner to a solid support, e.g., a microtiter plate or a paramagnetic microparticle.

In some embodiments, the solid support is a microtiter plate. In some embodiments, the solid support is a paramagnetic bead. An exemplary paramagnetic bead is DYNABEADS® MYONE™ Streptavidin C1 (Cat. Nos. 650.01-03). Other suitable beads will be apparent to those of skill in the art. Methods for attachment of antibodies to paramagnetic beads are well-known in the art.

The molecule of interest is contacted with the capture binding partner, e.g., capture antibody immobilized on a solid support. Some sample preparation may be used; e.g., preparation of serum from blood samples or concentration procedures before the sample is contacted with the capture antibody. Protocols for binding of proteins in immunoassays are well-known in the art and are included in the Examples.

The time allowed for binding will vary depending on the conditions; it will be apparent that shorter binding times are desirable in some settings, especially in a clinical setting. The use of, e.g., paramagnetic beads can reduce the time required for binding. In some embodiments, the time allowed for binding of the molecule of interest to the capture binding partner, e.g., an antibody, is less that about 12, 10, 8, 6, 4, 3, 2, or 1 hours, or less than about 60, 50, 40, 30, 25, 20, 15, 10, or 5 minutes.

In some embodiments, following the binding of particles of the molecule of interest to the capture binding partner, e.g., a capture antibody, particles that bound nonspecifically, as well as other unwanted substances in the sample, are washed away leaving substantially only specifically bound particles of the molecule of interest. In other embodiments, no wash is used between additions of sample and label, which can reduce sample preparation time. Thus, in some embodiments, the time allowed for both binding of the molecule of interest to the capture binding partner, e.g., an antibody, and binding of the label to the molecule of interest, is less that about 12, 10, 8, 6, 4, 3, 2, or 1 hours, or less than about 60, 50, 40, 30, 25, 20, 15, 10, or 5 minutes.

Label is added either with or following the addition of sample and washing. Protocols for binding antibodies and other immunolabels to proteins and other molecules are well-known in the art. If the label binding step is separate from that of capture binding, the time allowed for label binding can be important, e.g., in clinical applications or other time sensitive settings. In some embodiments, the time allowed for binding of the molecule of interest to the label, e.g., an antibody-dye, is less than about 12, 10, 8, 6, 4, 3, 2, or 1 hours, or less than about 60, 50, 40, 30, 25, 20, 15, 10, or 5 minutes. Excess label is removed by washing.

In another embodiment, the solid phase binding assay can be a competitive binding assay. One such method is as follows. First, a capture antibody immobilized on a binding surface is competitively bound by i) a molecule of interest, e.g., marker of a biological state, in a sample, and ii) a labeled analog of the molecule comprising a detectable label (the detection reagent). Second, the amount of the label using a single molecule analyzer is measured. Another such method is as follows. First, an antibody having a detectable label (the detection reagent) is competitively bound to i) a molecule of interest, e.g., marker of a biological state in a sample, and ii) an analog of the molecule that is immobilized on a binding surface (the capture reagent). Second, the amount of the label using a single molecule analyzer is measured. An “analog of a molecule” refers, herein, to a species that competes with a molecule for binding to a capture antibody.

In some embodiments, the label is not eluted from the protein of interest. In other embodiments, the label is eluted from the protein of interest. Preferred elution buffers are effective in releasing the label without generating significant background. It is useful if the elution buffer is bacteriostatic. Elution buffers used in the invention can include a chaotrope, a buffer, an albumin to coat the surface of the microtiter plate, and a surfactant selected so as to produce a relatively low background. The chaotrope can include urea, a guanidinium compound, or other useful chaotropes. The buffer can include borate buffered saline, or other useful buffers. The protein carrier can include, e.g., an albumin, such as human, bovine, or fish albumin, an IgG, or other useful carriers. The surfactant can include an ionic or nonionic detergent including Tween 20, Triton X-100, sodium dodecyl sulfate (SDS), and others.

Following elution, the label is run through a single molecule detector in, e.g., the elution buffer. A processing sample may contain no label, a single label, or a plurality of labels. The number of labels corresponds or is proportional to (if dilutions or fractions of samples are used) the number of molecules of the molecule of interest, e.g., marker of a biological state captured during the capture step.

Any suitable single molecule detector capable of detecting the label used with the molecule of interest may be used. Examples, of suitable single molecule detectors are described herein. Typically the detector will be part of a system that includes an automatic sampler for sampling prepared samples, and, optionally, a recovery system to recover samples.

In some embodiments, the processing sample is analyzed in a single molecule analyzer that utilizes a capillary flow system, and that includes a capillary flow cell, an electromagnetic radiation source to illuminate an interrogation space in the capillary through which processing sample is passed, a detector to detect radiation emitted from the interrogation space, and a source of motive force to move a processing sample through the interrogation space. In some embodiments, the single molecule analyzer further includes a microscope objective lens that collects light emitted from processing sample as it passes through the interrogation space, e.g., a high numerical aperture microscope objective. In some embodiments, the laser and detector are in a confocal arrangement. In some embodiments, the laser is a continuous wave laser. In some embodiments, the detector is an avalanche photodiode detector. In some embodiments, the source of motive force is a pump to provide pressure. In some embodiments, the invention provides an analyzer system that includes a sampling system capable of automatically sampling a plurality of samples providing a fluid communication between a sample container and the interrogation space.

In some embodiments, the interrogation space has a volume of between about 0.001 and 500 pL, or between about 0.01 pL and 100 pL, or between about 0.01 pL and 10 pL, or between about 0.01 pL and 5 pL, or between about 0.01 pL and 0.5 pL. Similarly the interrogation space has a volume between about 0.02 pL and about 300 pL, or between about 0.02 pL and about 50 pL or between about 0.02 pL and about 5 pL or between about 0.02 pL and about 0.5 pL or between about 0.02 pL and about 2 pL, or between about 0.05 pL and about 50 pL, or between about 0.05 pL and about 5 pL, or between about 0.05 pL and about 0.5 pL, or between about 0.05 pL and about 0.2 pL, or between about 0.1 pL and about 25 pL. For example, between about 0.004 pL and 100 pL, between about 0.02 pL and 50 pL, between about 0.001 pL and 10 pL, between about 0.001 pL and 10 pL, between about 0.01 pL and 5 pL, between about 0.02 pL and about 5 pL, between about 0.05 pL and 5 pL, between about 0.05 pL and 10 pL, between about 0.5 pL and about 5 pL, or between about 0.02 pL and about 0.5 pL.

In some embodiments, the interrogation space has a volume of more than about 1 μm³, more than about 2 μm³, for example, more than about 3 μm³, 4 μm³, 5 μm³, 10 μm³, 15 μm³, 30 μm³, 50 μm³, 75 μm³, 100 μm³, 150 μm³, 200 μm³, 250 μm³, 300 μm³, 1400 μm³, 500 μm³, 550 μm³, 600 μm³, 750 μm³, 1000 μm³, 2000 μm³, 4000 μm³, 6000 μm³, 8000 μm³, 10000 μm³, 12000 μm³, 13000 μm³, 14000 μm³, 15000 μm³, 20000 μm³, 30000 μm³, 40000 μm³, or more than about 50000 μm³. In some embodiments, the interrogation space is of a volume less than about 50000 μm³, for example, less than about 40000 μm³, 30000 μm³, 20000 μm³, 15000 μm³, 14000 μm³, 13000 μm³, 12000 μm³, less 11000 μm³, 9500 μm³, 8000 μm³, 6500 μm³, 6000 μm³, 5000 μm³, 4000 μm³, 3000 μm³, 2500 μm³, 2000 μm³, 1500 μm³, 1000 μm³, 800 μm³, 600 μm³, 400 μm³, 200 μm³, 100 μm³, 75 μm³, 50 μm³, 25 μm³, 20 μm³, 15 μm³, 14 μm³, 13 μm³, 12 μm³, 11 μm³, 10 μm³, 5 μm³, 4 μm³, μm³, less than about 2 μm³, or less than about 1 μm³. In some embodiments, the volume of the interrogation space is between about 1 μm³ and about 10000 μm³, between about 1 μm³ and about 1000 μm³, between about 1 μm³ and about 100 μm³ between about 1 μm³ and about 50 μm³ between about 1 μm³ and about 10 μm³, 2 μm³ and about 10 μm³ or between about 3 μm³ and about 7 μm³.

In some embodiments, an example of a single molecule detector used in the methods of the invention utilizes a capillary flow system, and includes a capillary flow cell, a continuous wave laser to illuminate an interrogation space in the capillary through which processing sample is passed, a high numerical aperture microscope objective lens, for example at least about 0.8, that collects light emitted from processing sample as it passes through the interrogation space, an avalanche photodiode detector to detect radiation emitted from the interrogation space, and a pump to provide pressure to move a processing sample through the interrogation space. In any of these embodiments the analyzer does not contain more than one interrogation space.

In some embodiments, the single molecule detector includes a scanning analyzer system, as disclosed in U.S. Patent Publication No. 2009/0159812 and entitled “Scanning Analyzer for Single Molecule Detection and Methods of Use.” In some embodiments, the single molecule detector used in the methods of the invention uses a sample plate, a continuous wave laser directed toward a sample plate in which the sample is contained, a high numerical aperture microscope objective lens that collects light emitted from the sample as interrogation space is translated through the sample, wherein the lens has a numerical aperture of at least about 0.8, an avalanche photodiode detector to detect radiation emitted from the interrogation space, and a scan motor with a moveable mirror to translate the interrogation space. In any of these embodiments, the analyzer can contain only one interrogation space.

In some embodiments, the single molecule detector is capable of determining a concentration for a molecule of interest in a sample where sample may range in concentration over a range of at least about 100-fold, or 1000-fold, or 10.000-fold, or 100.000-fold, or 300.00-fold, or 1,000,000-fold, or 10,000,000-fold, or 30,000,000-fold.

In some embodiments, the methods of the invention utilize a single molecule detector capable detecting a difference of less than about 50%, 40%, 30%, 20%, 15%, or 10% in concentration of an analyte between a first sample and a second sample that are introduced into the detector, where the volume of the first sample and said second sample introduced into the analyzer is less than about 100, 90, 80, 70, 60, 50, 40, 30, 20, 15, 10, 5, 4, 3, 2, or 1 μl, and wherein the analyte is present at a concentration of less than about 100, 90, 80, 70, 60, 50, 40, 30, 20, 15, 10, 5, 4, 3, 2, or 1 femtomolar.

The single molecule detector and systems are described in more detail below. Further embodiments of single molecule analyzers useful in the methods of the invention, such as detectors with more than one interrogation window, detectors utilize electrokinetic or electrophoretic flow, and the like, may be found in U.S. Pat. No. 7,572,640.

Between runs the instrument may be washed. A wash buffer that maintains the salt and surfactant concentrations of the sample may be used in some embodiments to maintain the conditioning of the capillary; i.e., to keep the capillary surface relatively constant between samples to reduce variability.

A feature that contributes to the extremely high sensitivity of the instruments and methods of the invention is the method of detecting and counting labels, which, in some embodiments, are attached to single molecules to be detected or, more typically, correspond to a single molecule to be detected. Briefly, the processing sample flowing through the capillary or contained on a sample plate is effectively divided into a series of detection events, by subjecting a given interrogation space of the capillary to EM radiation from a laser that emits light at an appropriate excitation wavelength for the fluorescent moiety used in the label for a predetermined period of time, and detecting photons emitted during that time. Each predetermined period of time is a “bin.” If the total number of photons detected in a given bin exceeds a predetermined threshold level, a detection event is registered for that bin, i.e., a label has been detected. If the total number of photons is not at the predetermined threshold level, no detection event is registered. In some embodiments, processing sample concentration is dilute enough that, for a large percentage of detection events, the detection event represents only one label passing through the window, which corresponds to a single molecule of interest in the original sample, that is, few detection events represent more than one label in a single bin. In some embodiments, further refinements are applied to allow greater concentrations of label in the processing sample to be detected accurately, i.e., concentrations at which the probability of two or more labels being detected as a single detection event is no longer insignificant.

Although other bin times can be used without departing from the scope of the present invention, in some embodiments the bin times are selected in the range of about 1 microsecond to about 5 ms. In some embodiments, the bin time is more than about 1, 2, 3, 4, 5, 6, 7, 8, 9, 10, 15, 20, 30, 40, 50, 60, 70, 80, 90, 100, 200, 250, 300, 400, 500, 600, 700, 750, 800, 900, 1000, 2000, 3000, 4000, or 5000 microseconds. In some embodiments, the bin time is less than about 2, 3, 4, 5, 6, 7, 8, 9, 10, 15, 20, 30, 40, 50, 60, 70, 80, 90, 100, 200, 250, 300, 400, 500, 600, 700, 750, 800, 900, 1000, 2000, 3000, 4000, or 5000 microseconds.

In some embodiments, determining the concentration of a particle-label complex in a sample includes determining the background noise level. In some embodiments, the background noise level is determined from the mean noise level, or the root-mean-square noise. In other cases, a typical noise value or a statistical value is chosen. In most cases, the noise is expected to follow a Poisson distribution.

Thus, as a label is encountered in the interrogation space, it is irradiated by the laser beam to generate a burst of photons. The photons emitted by the label are discriminated from background light or background noise emission by considering only the bursts of photons that have energy above a predetermined threshold energy level which accounts for the amount of background noise that is present in the sample. Background noise typically includes low frequency emission produced, for example, by the intrinsic fluorescence of non-labeled particles that are present in the sample, the buffer or diluent used in preparing the sample for analysis, Raman scattering and electronic noise. In some embodiments, the value assigned to the background noise is calculated as the average background signal noise detected in a plurality of bins, which are measurements of photon signals that are detected in an interrogation space during a predetermined length of time. Thus in some embodiments, background noise is calculated for each sample as a number specific to that sample.

Given the value for the background noise, the threshold energy level can be assigned. As discussed above, the threshold value is determined to discriminate true signals (due to fluorescence of a label) from the background noise. Care must be taken in choosing a threshold value such that the number of false positive signals from random noise is minimized while the number of true signals which are rejected is also minimized. Methods for choosing a threshold value include determining a fixed value above the noise level and calculating a threshold value based on the distribution of the noise signal. In one embodiment, the threshold is set at a fixed number of standard deviations above the background level. Assuming a Poisson distribution of the noise, using this method one can estimate the number of false positive signals over the time course of the experiment. In some embodiments, the threshold level is calculated as a value of 4 sigma above the background noise. For example, given an average background noise level of 200 photons, the analyzer system establishes a threshold level of 4-000 above the average background/noise level of 200 photons to be 256 photons. Thus, in some embodiments, determining the concentration of a label in a sample includes establishing the threshold level above which photon signals represent the presence of a label. Conversely, photon signals that have an energy level that is not greater than that of the threshold level indicate the absence of a label.

Many bin measurements are taken to determine the concentration of a sample, and the absence or presence of a label is ascertained for each bin measurement. Typically, 60,000 measurements or more can made in one minute (e.g., in embodiments in which the bin size is 1 ms—for smaller bin sizes the number of measurements is correspondingly larger, e.g., 6,000,000 measurements per minute for a bin size of 10 microseconds). Thus, no single measurement is crucial and the method provides for a high margin of error. The bins that are determined not to contain a label (“no” bins) are discounted and only the measurements made in the bins that are determined to contain label (“yes” bins) are accounted in determining the concentration of the label in the processing sample. Discounting measurements made in the “no” bins or bins that are devoid of label increases the signal to noise ratio and the accuracy of the measurements. Thus, in some embodiments, determining the concentration of a label in a sample includes detecting the bin measurements that reflect the presence of a label.

The signal to noise ratio or the sensitivity of the analyzer system can be increased by minimizing the time that background noise is detected during a bin measurement in which a particle-label complex is detected. For example, in a bin measurement lasting 1 millisecond during which one particle-label complex is detected when passing across an interrogation space within 250 microseconds, 750 microseconds of the 1 millisecond are spent detecting background noise emission. The signal to noise ratio can be improved by decreasing the bin time. In some embodiments, the bin time is 1 millisecond. In other embodiments, the bin time is 750, 500, 250 microseconds, 100 microseconds, 50 microseconds, 25 microseconds or 10 microseconds. Other bin times are as described herein.

Other factors that affect measurements are the brightness or dimness of the fluorescent moiety, the flow rate, and the power of the laser. Various combinations of the relevant factors that allow for detection of label will be apparent to those of skill in the art. In some embodiments, the bin time is adjusted without changing the flow rate. It will be appreciated by those of skill in the art that as bin time decreases, laser power output directed at the interrogation space must increase to maintain a constant total energy applied to the interrogation space during the bin time. For example, if bin time is decreased from 1000 microseconds to 250 microseconds, as a first approximation, laser power output must be increased approximately four-fold. These settings allow for the detection of the same number of photons in a 250 μs as the number of photons counted during the 1000 μs given the previous settings, and allow for faster analysis of sample with lower backgrounds and thus greater sensitivity. In addition, flow rates may be adjusted in order to speed processing of sample. These numbers are merely exemplary, and the skilled practitioner can adjust the parameters as necessary to achieve the desired result.

In some embodiments, the interrogation space encompasses the entire cross-section of the sample stream. When the interrogation space encompasses the entire cross-section of the sample stream, only the number of labels counted and the volume passing through a cross-section of the sample stream in a set length of time are needed to calculate the concentration of the label in the processing sample. In some embodiments, the interrogation space can be defined to be smaller than the cross-sectional area of sample stream by, for example, the interrogation space is defined by the size of the spot illuminated by the laser beam. In some embodiments, the interrogation space can be defined by adjusting the apertures of the analyzer and reducing the illuminated volume that is imaged by the objective lens to the detector. In the embodiments when the interrogation space is defined to be smaller than the cross-sectional area of sample stream, the concentration of the label can be determined by interpolation of the signal emitted by the complex from a standard curve that is generated using one or more samples of known standard concentrations. In yet other embodiments, the concentration of the label can be determined by comparing the measured particles to an internal label standard. In embodiments when a diluted sample is analyzed, the dilution factor is accounted in calculating the concentration of the molecule of interest in the starting sample.

As discussed above, when the interrogation space encompasses the entire cross-section of the sample stream, only the number of labels counted passing through a cross-section of the sample stream in a set length of time (bin) and the volume of sample that was interrogated in the bin are needed to calculate the concentration the sample. The total number of labels contained in the “yes” bins is determined and related to the sample volume represented by the total number of bins used in the analysis to determine the concentration of labels in the processing sample. Thus, in one embodiment, determining the concentration of a label in a processing sample includes determining the total number of labels detected “yes” bins and relating the total number of detected labels to the total sample volume that was analyzed. The total sample volume that is analyzed is the sample volume that is passed through the capillary flow cell and across the interrogation space in a specified time interval. Alternatively, the concentration of the label complex in a sample is determined by interpolation of the signal emitted by the label in a number of bins from a standard curve that is generated by determining the signal emitted by labels in the same number of bins by standard samples containing known concentrations of the label.

In some embodiments, the number of individual labels that are detected in a bin is related to the relative concentration of the particle in the processing sample. At relatively low concentrations, for example at concentrations below about 10⁻¹⁶ M the number of labels is proportional to the photon signal that is detected in a bin. Thus, at low concentrations of label the photon signal is provided as a digital signal. At relatively higher concentrations, for example at concentrations greater than about 10⁻¹⁶ M, the proportionality of photon signal to a label is lost as the likelihood of two or more labels crossing the interrogation space at about the same time and being counted as one becomes significant. Thus, in some embodiments, individual particles in a sample of a concentration greater than about 10⁻¹⁶ M are resolved by decreasing the length of time of the bin measurement.

Alternatively, in other embodiments, the total photon signal that is emitted by a plurality of particles that are present in any one bin is detected. These embodiments allow for single molecule detectors of the invention wherein the dynamic range is at least 3, 3.5, 4, 4.5, 5.5, 6, 6.5, 7, 7.5, 8, or more than 8 logs.

“Dynamic range,” as that term is used herein, refers to the range of sample concentrations that may be quantitated by the instrument without need for dilution or other treatment to alter the concentration of successive samples of differing concentrations, where concentrations are determined with an accuracy appropriate for the intended use. For example, if a microliter plate contains a sample of 1 femtomolar concentration for an analyte of interest in one well, a sample of 10,000 femtomolar concentration for an analyte of interest in another well, and a sample of 100 femtomolar concentration for the analyte in a third well, an instrument with a dynamic range of at least 4 logs and a lower limit of quantitation of 1 femtomolar is able to accurately quantitate the concentration of all the samples without the need for further treatment to adjust concentration, e.g., dilution. Accuracy may be determined by standard methods, e.g., using a series of standards of concentrations that span the dynamic range and constructing a standard curve. Standard measures of fit of the resulting standard curve may be used as a measure of accuracy, e.g., an r² greater than about 0.7, 0.75, 0.8, 0.85, 0.9, 0.91, 0.92, 0.93, 0.94, 0.95, 0.96, 0.97, 0.98, or 0.99.

Increased dynamic range is achieved by altering the manner in which data from the detector is analyzed, and/or by the use of an attenuator between the detector and the interrogation space. At the low end of the range, where processing sample is sufficiently dilute that each detection event, i.e., each burst of photons above a threshold level in a bin (the “event photons”), likely represents only one label, the data is analyzed to count detection events as single molecules. Thereby each bin is analyzed as a simple “yes” or “no” for the presence of label, as described above. For a more concentrated processing sample, where the likelihood of two or more labels occupying a single bin becomes significant, the number of event photons in a significant number of bins is found to be substantially greater than the number expected for a single label, e.g., the number of event photons in a significant number of bins corresponds to two-fold, three-fold, or more, than the number of event photons expected for a single label. For these samples, the instrument changes its method of data analysis to one of integrating the total number of event photons for the bins of the processing sample. This total will be proportional to the total number of labels that were in all the bins. For an even more concentrated processing sample, where many labels are present in most bins, background noise becomes an insignificant portion of the total signal from each bin, and the instrument changes its method of data analysis to one of counting total photons per bin (including background). An even further increase in dynamic range can be achieved by the use of an attenuator between the flow cell and the detector, when concentrations are such that the intensity of light reaching the detector would otherwise exceed the capacity of the detector for accurately counting photons, i.e., saturate the detector.

The instrument may include a data analysis system that receives input from the detector and determines the appropriate analysis method for the sample being run, and outputs values based on such analysis. The data analysis system may further output instructions to use or not use an attenuator, if an attenuator is included in the instrument.

By utilizing such methods, the dynamic range of the instrument can be dramatically increased. Thus, in some embodiments, the instrument is capable of measuring concentrations of samples over a dynamic range of more than about 1000 (3 log), 10,000 (4 log), 100,000 (5 log), 350,000 (5.5 log), 1,000,000 (6 log), 3,500,000 (6.5 log), 10,000,000 (7 log), 35,000,000 (7.5 log), or 100,000,000 (8 log). In some embodiments, the instrument is capable of measuring concentrations of samples over a dynamic range of more than about 100,000 (5 log). In some embodiments, the instrument is capable of measuring concentrations of samples over a dynamic range of more than about 1,000,000 (6 log). In some embodiments, the instrument is capable of measuring concentrations of samples over a dynamic range of more than about 10,000,000 (7 log). In some embodiments, the instrument is capable of measuring the concentrations of samples over a dynamic range of from about 1-10 femtomolar to at least about 1000; 10,000; 100,000; 350,000; 1,000,000; 3,500,000; 10,000,000; or 35,000,000 femtomolar. In some embodiments, the instrument is capable of measuring the concentrations of samples over a dynamic range of from about 1-10 femtomolar to at least about 10,000 femtomolar. In some embodiments, the instrument is capable of measuring the concentrations of samples over a dynamic range of from about 1-10 femtomolar to at least about 100,000 femtomolar. In some embodiments, the instrument is capable of measuring the concentrations of samples over a dynamic range of from about 1-10 femtomolar to at least about 1,000,000 femtomolar. In some embodiments, the instrument is capable of measuring the concentrations of samples over a dynamic range of from about 1-10 femtomolar to at least about 10,000,000.

In some embodiments, an analyzer or analyzer system of the invention is capable of detecting an analyte, e.g., a biomarker, at a limit of detection of less than 1 nanomolar, or 1 picomolar, or 1 femtomolar, or 1 attomolar, or 1 zeptomolar. In some embodiments, the analyzer or analyzer system is capable of detecting a change in concentration of the analyte, or of multiple analytes, e.g., a biomarker or biomarkers, from one sample to another sample of less than about 0.1%, 1%, 2%, 5%, 10%, 20%, 30%, 40%, 50%, 60%, 70% or 80% when the biomarker is present at a concentration of less than 1 nanomolar, or 1 picomolar, or 1 femtomolar, or 1 attomolar, or 1 zeptomolar, in the samples, and when the size of each of the sample is less than about 100, 50, 40, 30, 20, 10, 5, 2, 1, 0.1, 0.01, 0.001, or 0.0001 μl. In some embodiments, the analyzer or analyzer system is capable of detecting a change in concentration of the analyte from a first sample to a second sample of less than about 20%, when the analyte is present at a concentration of less than about 1 picomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 100 femtomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 50 femtomolar, and when the size of each of the samples is less than about In some embodiments, the analyzer or analyzer system is capable of detecting a change in concentration of the analyte from a first sample to a second sample of less than about 20%, when the analyte is present at a concentration of less than about 5 femtomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 5 femtomolar, and when the size of each of the samples is less than about 5 μl. In some embodiments, the analyzer or analyzer system is capable of detecting a change in concentration of the analyte from a first sample to a second sample of less than about 20%, when the analyte is present at a concentration of less than about 1 femtomolar, and when the size of each of the samples is less than about 5 μl.

The single molecule detectors of the present invention are capable of detecting molecules of interest in a highly sensitive manner with a very low coefficient of variation (CV). In some embodiments, the CV is less than about 50%, 40%, 30%, 25%, 20%, 15%, 10%, 5%, or less than about 1%. In various example, the LOD is less than 100, 50, 40, 50, 20, 15, 10, 5, 1, 0.5, or 0.1 pg/ml, and the CV is less than about 10%, about 5%, or about 1%.

In one embodiment, the analyzer system is capable of single molecule detection of a fluorescently labeled particle wherein the analyzer system detects energy emitted by an excited fluorescent label in response to exposure by an electromagnetic radiation source when the single particle is present in an interrogation space defined within a capillary flow cell fluidly connected to the sampling system of the analyzer system. In a further embodiment of the analyzer system, the single particle moves through the interrogation space of the capillary flow cell by means of a motive force. In another embodiment of the analyzer system, an automatic sampling system may be included in the analyzer system for introducing the sample into the analyzer system. In another embodiment of the analyzer system, a sample preparation system may be included in the analyzer system for preparing a sample. In a further embodiment, the analyzer system may contain a sample recovery system for recovering at least a portion of the sample after analysis is complete.

In one aspect, the analyzer system consists of an electromagnetic radiation source for exciting a single particle labeled with a fluorescent label. In one embodiment, the electromagnetic radiation source of the analyzer system is a laser. In a further embodiment, the electromagnetic radiation source is a continuous wave laser.

In a typical embodiment, the electromagnetic radiation source excites a fluorescent moiety attached to a label as the label passes through the interrogation space of the capillary flow cell. In some embodiments, the fluorescent label moiety includes one or more fluorescent dye molecules. In some embodiments, the fluorescent label moiety is a quantum dot. Any fluorescent moiety as described herein may be used in the label.

A label is exposed to electromagnetic radiation when the label passes through an interrogation space located within the capillary flow cell. The interrogation space is typically fluidly connected to a sampling system. In some embodiments the label passes through the interrogation space of the capillary flow cell due to a motive force to advance the label through the analyzer system. The interrogation space is positioned such that it receives electromagnetic radiation emitted from the radiation source. In some embodiments, the sampling system is an automated sampling system capable of sampling a plurality of samples without intervention from a human operator.

The label passes through the interrogation space and emits a detectable amount of energy when excited by the electromagnetic radiation source. In one embodiment, an electromagnetic radiation detector is operably connected to the interrogation space. The electromagnetic radiation detector is capable of detecting the energy emitted by the label, e.g., by the fluorescent moiety of the label.

In a further embodiment of the analyzer system, the system further includes a sample preparation mechanism where a sample may be partially or completely prepared for analysis by the analyzer system. In some embodiments of the analyzer system, the sample is discarded after it is analyzed by the system. In other embodiments, the analyzer system further includes a sample recovery mechanism whereby at least a portion, or alternatively all or substantially all, of the sample may be recovered after analysis. In such an embodiment, the sample can be returned to the origin of the sample. In some embodiments, the sample can be returned to microtiter wells on a sample microtiter plate. The analyzer system typically further consists of a data acquisition system for collecting and reporting the detected signal.

The analyzer system can include an electromagnetic radiation source, a mirror, a lens, a capillary flow cell, a microscopic objective lens, an aperture, a detector lens, a detector filter, a single photon detector, and a processor operatively connected to the detector.

In operation the electromagnetic radiation source is aligned so that its output is reflected off of a front surface of the mirror. The lens focuses the beam onto a single interrogation space in the capillary flow cell. The microscope objective lens collects light from sample particles and forms images of the beam onto the aperture. The aperture affects the fraction of light emitted by the specimen in the interrogation space of the capillary flow cell that can be collected. The detector lens collects the light passing through the aperture and focuses the light onto an active area of the detector after it passes through the detector filters. The detector filters minimize aberrant noise signals due to light scatter or ambient light while maximizing the signal emitted by the excited fluorescent moiety bound to the particle. The processor processes the light signal from the particle according to the methods described herein.

In one embodiment, the microscope objective lens is a high numerical aperture microscope objective. As used herein, “high numerical aperture lens” include a lens with a numerical aperture of equal to or greater than 0.6. The numerical aperture is a measure of the number of highly diffracted image-forming light rays captured by the objective. A higher numerical aperture allows increasingly oblique rays to enter the objective lens and thereby produce a more highly resolved image. Additionally, the brightness of an image increases with a higher numerical aperture. High numerical aperture lenses are commercially available from a variety of vendors, and any one lens having a numerical aperture of equal to or greater than approximately 0.6 may be used in the analyzer system. In some embodiments, the lens has a numerical aperture of about 0.6 to about 1.3. In some embodiments, the aperture of the microscope objective lens 305 is approximately 1.25. In an embodiment where a microscope objective lens 305 of 0.8 is used, a Nikon 60×/0.8 NA Achromat lens (Nikon, Inc., USA) can be used.

In some embodiments, the electromagnetic radiation source is a laser that emits light in the visible spectrum. In all embodiments, the electromagnetic radiation source is set such that wavelength of the laser is set such that it is of a sufficient wavelength to excite the fluorescent label attached to the particle. In some embodiments, the laser is a continuous wave laser with a wavelength of 639 nm. In other embodiments, the laser is a continuous wave laser with a wavelength of 532 nm. In other embodiments, the laser is a continuous wave laser with a wavelength of 422 nm. In other embodiments, the laser is a continuous wave laser with a wavelength of 405 nm. Any continuous wave laser with a wavelength suitable for exciting a fluorescent moiety as used in the methods and compositions of the invention may be used without departing from the scope of the invention.

In a single particle analyzer system, as each particle passes through the beam of the electromagnetic radiation source, the particle enters into an excited state. When the particle relaxes from its excited state, a detectable burst of light is emitted. The excitation-emission cycle is repeated many times by each particle in the length of time it takes for it to pass through the beam allowing the analyzer system to detect tens to thousands of photons for each particle as it passes through an interrogation space. Photons emitted by fluorescent particles are registered by the detector with a time delay indicative of the time for the particle label complex to pass through the interrogation space. The photon intensity is recorded by the detector and sampling time is divided into bins, which are uniform, arbitrary, time segments with freely selectable time channel widths. The number of signals contained in each bin evaluated. One or a combination of several statistical analytical methods are employed in order to determine when a particle is present. Such methods include determining the baseline noise of the analyzer system and setting a signal strength for the fluorescent label at a statistical level above baseline noise to eliminate false positive signals from the detector.

The electromagnetic radiation source is focused onto a capillary flow cell of the analyzer system 300 where the capillary flow cell is fluidly connected to the sample system. The beam from the continuous wave electromagnetic radiation source is optically focused to a specified depth within the capillary flow cell. The beam is directed toward the sample-filled capillary flow cell at an angle perpendicular to the capillary flow cell. The beam is operated at a predetermined wavelength that is selected to excite a particular fluorescent label used to label the particle of interest. The size or volume of the interrogation space is determined by the diameter of the beam together with the depth at which the beam is focused. Alternatively, the interrogation space can be determined by running a calibration sample of known concentration through the analyzer system.

When single molecules are detected in the sample concentration, the beam size and the depth of focus required for single molecule detection are set and thereby define the size of the interrogation space. The interrogation space is set such that, with an appropriate sample concentration, only one particle is present in the interrogation space during each time interval over which time observations are made.

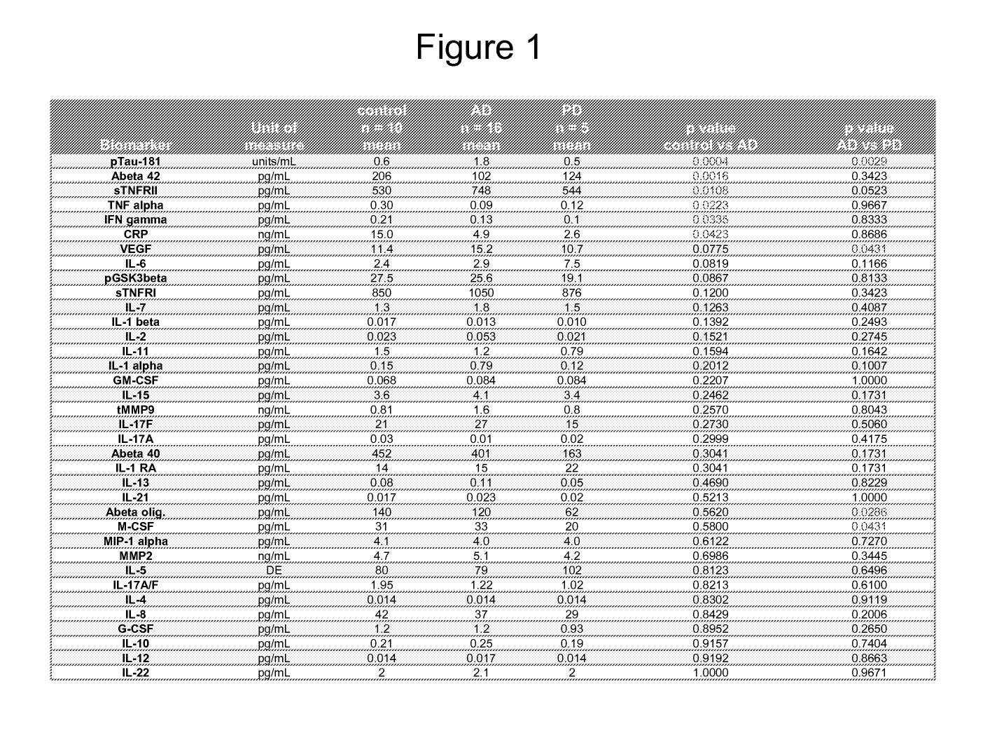

It will be appreciated that the detection interrogation volume as defined by the beam is not perfectly spherically shaped, and typically is a “bow-tie” shape. However, for the purposes of definition, “volumes” of interrogation spaces are defined herein as the volume encompassed by a sphere of a diameter equal to the focused spot diameter of the beam. The focused spot of the beam may have various diameters without departing from the scope of the present invention. In some embodiments, the diameter of the focused spot of the beam is about 1 to about 5, 10, 15, or 20 microns, or about 5 to about 10, 15, or 20 microns, or about 10 to about 20 microns, or about 10 to about 15 microns. In some embodiments, the diameter of the focused spot of the beam is about 1, 2, 3, 4, 5, 6, 7, 8, 9, 10, 11, 12, 13, 14, 15, 16, 17, 18, 19, or 20 microns. In an alternate embodiment of the single particle analyzer system, more than one electromagnetic radiation source can be used to excite particles labeled with fluorescent labels of different wavelengths. In another alternate embodiment, more than one interrogation space in the capillary flow cell can be used. In another alternate embodiment, multiple detectors can be employed to detect different emission wavelengths from the fluorescent labels. An illustration incorporating each of these alternative embodiments of an analyzer system is shown in FIG. 1B of U.S. Patent Publication No. 2006/0078998.

In some embodiments of the analyzer system, a motive force is required to move a particle through the capillary flow cell of the analyzer system. In one embodiment, the motive force can be a form of pressure. The pressure used to move a particle through the capillary flow cell can be generated by a pump. In some embodiments, a Scivex, Inc. HPLC pump can be used. In some embodiments where a pump is used as a motive force, the sample can pass through the capillary flow cell at a rate of 1 μL/min to about 20 μL/min, or about 5 μL/min to about 20 μL/min. In some embodiments, the sample can pass through the capillary flow cell at a rate of about 5 μL/min. In some embodiments, the sample can pass through the capillary flow cell at a rate of about 10 μL/min. In some embodiments, the sample can pass through the capillary flow cell at a rate of about 15 μL/min. In some embodiments, the sample can pass through the capillary flow cell at a rate of about 20 μL/min. In some embodiments, an electrokinetic force can be used to move the particle through the analyzer system. Such a method has been previously disclosed in U.S. Pat. No. 7,572,640.

In one aspect of the analyzer system, the detector of the analyzer system detects the photons emitted by the fluorescent label. In one embodiment, the photon detector is a photodiode. In a further embodiment, the detector is an avalanche photodiode detector. In some embodiments, the photodiodes can be silicon photodiodes with a wavelength detection of 190 nm and 1100 nm. When germanium photodiodes are used, the wavelength of light detected is between 400 nm to 1700 nm. In other embodiments, when an indium gallium arsenide photodiode is used, the wavelength of light detected by the photodiode is between 800 nm and 2600 nm. When lead sulfide photodiodes are used as detectors, the wavelength of light detected is between 1000 nm and 3500 nm.

In some embodiments, the optics of the electromagnetic radiation source and the optics of the detector are arranged in a conventional optical arrangement. In such an arrangement, the electromagnetic radiation source and the detector are aligned on different focal planes. The arrangement of the laser and the detector optics of the analyzer system is that of a conventional optical arrangement.

In some embodiments, the optics of the electromagnetic radiation source and the optics of the detector are arranged in a confocal optical arrangement. In such an arrangement, the electromagnetic radiation source and the detector are aligned on the same focal plane. The confocal arrangement renders the analyzer more robust because the electromagnetic radiation source and the detector optics do not need to be realigned if the analyzer system is moved. This arrangement also makes the use of the analyzer more simplified because it eliminates the need to realign the components of the analyzer system. The beam from an electromagnetic radiation source is focused by the microscope objective to form one interrogation space within the capillary flow cell. A dichroic mirror, which reflects laser light but passes fluorescent light, is used to separate the fluorescent light from the laser light. Filter that is positioned in front of the detector eliminates any non-fluorescent light at the detector. In some embodiments, an analyzer system configured in a confocal arrangement can include two or more interrogations spaces. Such a method has been previously disclosed and is incorporated by reference from previous U.S. Pat. No. 7,572,640.

The laser can be a tunable dye laser, such as a helium-neon laser. The laser can be set to emit a wavelength of 632.8 nm. Alternatively, the wavelength of the laser can be set to emit a wavelength of 543.5 nm or 1523 nm. Alternatively, the electromagnetic laser can be an argon ion laser. In such an embodiment, the argon ion laser can be operated as a continuous gas laser at about 25 different wavelengths in the visible spectrum, the wavelength set between 408.9 and 686.1 nm but at its optimum performance set between 488 and 514.5 nm.

1. Electromagnetic Radiation Source

In some embodiments of the analyzer system a chemiluminescent label may be used. In such an embodiment, it may not be necessary to utilize an EM source for detection of the particle. In another embodiment, the extrinsic label or intrinsic characteristic of the particle is a light-interacting label or characteristic, such as a fluorescent label or a light-scattering label. In such an embodiment, a source of EM radiation is used to illuminate the label and/or the particle. EM radiation sources for excitation of fluorescent labels are preferred.

In some embodiments, the analyzer system consists of an electromagnetic radiation source. Any number of radiation sources may be used in any one analyzer system 300 without departing from the scope of the invention. Multiple sources of electromagnetic radiation have been previously disclosed and are incorporated by reference from previous U.S. Pat. No. 7,572,640. In some embodiments, all the continuous wave electromagnetic (EM) radiation sources emit electromagnetic radiation at the same wavelengths. In other embodiments, different sources emit different wavelengths of EM radiation.

In one embodiment, the EM source(s) are continuous wave lasers producing wavelengths of between 200 nm and 1000 nm. Such EM sources have the advantage of being small, durable and relatively inexpensive. In addition, they generally have the capacity to generate larger fluorescent signals than other light sources. Specific examples of suitable continuous wave EM sources include, but are not limited to: lasers of the argon, krypton, helium-neon, helium-cadmium types, as well as, tunable diode lasers (red to infrared regions), each with the possibility of frequency doubling. The lasers provide continuous illumination with no accessory electronic or mechanical devices, such as shutters, to interrupt their illumination. In an embodiment where a continuous wave laser is used, an electromagnetic radiation source of 3 mW may be of sufficient energy to excite a fluorescent label. A beam from a continuous wave laser of such energy output may be between 2 to 5 μm in diameter. The time of exposure of the particle to laser beam in order to be exposed to 3 mW may be a time period of about 1 msec. In alternate embodiments, the time of exposure to the laser beam may be equal to or less than about 500 μsec. In an alternate embodiment, the time of exposure may be equal to or less than about 100 μsec. In an alternate embodiment, the time of exposure may be equal to or less than about 50 μsec. In an alternate embodiment, the time of exposure may be equal to or less than about 10 μsec.

LEDs are another low-cost, high reliability illumination source. Recent advances in ultra-bright LEDs and dyes with high absorption cross-section and quantum yield support the applicability of LEDs to single particle detection. Such lasers could be used alone or in combination with other light sources such as mercury arc lamps, elemental arc lamps, halogen lamps, arc discharges, plasma discharges, light-emitting diodes, or combination of these.

In other embodiments, the EM source could be in the form of a pulse wave laser. In such an embodiment, the pulse size of the laser is an important factor. In such an embodiment, the size, focus spot, and the total energy emitted by the laser is important and must be of sufficient energy as to be able to excite the fluorescent label. When a pulse laser is used, a pulse of longer duration may be required. In some embodiments a laser pulse of 2 nanoseconds may be used. In some embodiments a laser pulse of 5 nanoseconds may be used. In some embodiments a pulse of between 2 to 5 nanoseconds may be used.

The optimal laser intensity depends on the photo bleaching characteristics of the single dyes and the length of time required to traverse the interrogation space (including the speed of the particle, the distance between interrogation spaces if more than one is used and the size of the interrogation space(s)). To obtain a maximal signal, it is desirable to illuminate the sample at the highest intensity which will not result in photo bleaching a high percentage of the dyes. The preferred intensity is one such that no more that 5% of the dyes are bleached by the time the particle has traversed the interrogation space.

The power of the laser is set depending on the type of dye molecules that need to be stimulated and the length of time the dye molecules are stimulated, and/or the speed with which the dye molecules pass through the capillary flow cell. Laser power is defined as the rate at which energy is delivered by the beam and is measured in units of Joules/second, or Watts. It will be appreciated that the greater the power output of the laser, the shorter the time that the laser illuminates the particle may be, while providing a constant amount of energy to the interrogation space while the particle is passing through the space. Thus, in some embodiments, the combination of laser power and time of illumination is such that the total energy received by the interrogation space during the time of illumination is more than about 0.1, 0.5, 1, 2, 3, 4, 5, 6, 7, 8, 9, 10, 12, 15, 20, 25, 30, 35, 40, 45, 50, 60, 70, 80, 90, or 100 microJoule. In some embodiments, the combination of laser power and time of illumination is such that the total energy received by the interrogation space during the time of illumination is less than about 0.5, 1, 2, 3, 4, 5, 6, 7, 8, 9, 10, 12, 15, 20, 25, 30, 35, 40, 45, 50, 60, 70, 80, 90, 100, or 110 microJoule.

In some embodiments, the laser power output is set to at least about 1 mW, 2 mW, 3 mW, 4 mW, 5 mW, 6, mw, 7 mW, 8 mW, 9 mW, 10 mW, 13 mW, 15 mW, 20 mW, 25 mW, 30 mW, 40 mW, 50 mW, 60 mW, 70 mW, 80 mW, 90 mW, 100 mW, or more than 100 mW. The time that the laser illuminates the interrogation space can be set to no less than about 1, 2, 3, 4, 5, 10, 15, 20, 30, 40, 50, 60, 70, 80, 90, 100, 150, 200, 150, 300, 350, 400, 450, 500, 600, 700, 800, 900, or 1000 microseconds. For example, the time that the laser illuminates the interrogation space can be set to 1 millisecond, 250 microseconds, 100 microseconds, 50 microseconds, 25 microseconds or 10 microseconds with a laser that provides a power output of 3 mW, 4 mw, 5 mW, or more than 5 mW. In some embodiments, a label is illuminated with a laser that provides a power output of 3 mW and illuminates the label for about 1000 microseconds. In other embodiments, a label is illuminated for less than 1000 milliseconds with a laser providing a power output of not more than about 20 mW. In other embodiments, the label is illuminated with a laser power output of 20 mW for less than or equal to about 250 microseconds. In some embodiments, the label is illuminated with a laser power output of about 5 mW for less than or equal to about 1000 microseconds.

When the system includes a capillary flow cell, it is fluidly connected to the sample system. In one embodiment, the interrogation space of an analyzer system is determined by the cross sectional area of the corresponding beam and by a segment of the beam within the field of view of the detector. In one embodiment of the analyzer system, the interrogation space has a volume, as defined herein, of between about between about 0.01 and 500 pL. Other useful interrogation space volumes are as described herein. It should be understood by one skilled in the art that the interrogation space can be selected for maximum performance of the analyzer. Although very small interrogation spaces have been shown to minimize the background noise, large interrogation spaces have the advantage that low concentration samples can be analyzed in a reasonable amount of time. In embodiments in which two interrogation spaces and are used, volumes such as those described herein for a single interrogation space may be used.

In one embodiment of the present invention, the interrogation spaces are large enough to allow for detection of particles at concentrations ranging from about 1000 femtomolar (fM) to about 1 zeptomolar (zM). In one embodiment of the present invention, the interrogation spaces are large enough to allow for detection of particles at concentrations ranging from about 1000 fM to about 1 attomolar (aM). In one embodiment of the present invention, the interrogation spaces are large enough to allow for detection of particles at concentrations ranging from about 10 fM to about 1 attomolar (aM). In many cases, the large interrogation spaces allow for the detection of particles at concentrations of less than about 1 fM without additional pre-concentration devices or techniques. One skilled in the art will recognize that the most appropriate interrogation space size depends on the brightness of the particles to be detected, the level of background signal, and the concentration of the sample to be analyzed.

The size of the interrogation space can be limited by adjusting the optics of the analyzer. In one embodiment, the diameter of the beam can be adjusted to vary the volume of the interrogation space. In another embodiment, the field of view of the detector can be varied. Thus, the source and the detector can be adjusted so that single particles will be illuminated and detected within the interrogation space. In another embodiment, the width of aperture that determine the field of view of the detector is variable. This configuration allows for altering the interrogation space, in near real time, to compensate for more or less concentrated samples, ensuring a low probability of two or more particles simultaneously being within an interrogation space. Similar alterations for two or more interrogation spaces may performed.

In another embodiment, the interrogation space can be defined through the use of a calibration sample of known concentration that is passed through the capillary flow cell prior to the actual sample being tested. When only one single particle is detected at a time in the calibration sample as the sample is passing through the capillary flow cell, the depth of focus together with the diameter of the beam of the electromagnetic radiation source determines the size of the interrogation space in the capillary flow cell.

Physical constraints to the interrogation spaces can also be provided by a solid wall. In one embodiment, the wall is one or more of the walls of a flow cell when the sample fluid is contained within a capillary. In one embodiment, the cell is made of glass, but other substances transparent to light in the range of about 200 to about 1,000 nm or higher, such as quartz, fused silica, and organic materials such as Teflon, nylon, plastics, such as polyvinylchloride, polystyrene, and polyethylene, or any combination thereof, may be used without departing from the scope of the present invention. Although other cross-sectional shapes (e.g., rectangular, cylindrical) may be used without departing from the scope of the present invention, in one embodiment the capillary flow cell has a square cross section. In another embodiment, the interrogation space may be defined at least in part by a channel (not shown) etched into a chip (not shown). Similar considerations apply to embodiments in which two interrogation spaces are used.

The interrogation space is bathed in a fluid. In one embodiment, the fluid is aqueous. In other embodiments, the fluid is non-aqueous or a combination of aqueous and non-aqueous fluids. In addition the fluid may contain agents to adjust pH, ionic composition, or sieving agents, such as soluble macroparticles or polymers or gels. It is contemplated that valves or other devices may be present between the interrogation spaces to temporarily disrupt the fluid connection. Interrogation spaces temporarily disrupted are considered to be connected by fluid.

In another embodiment of the invention, an interrogation space is the single interrogation space present within the flow cell which is constrained by the size of a laminar flow of the sample material within a diluent volume, also called sheath flow. In these and other embodiments, the interrogation space can be defined by sheath flow alone or in combination with the dimensions of the illumination source or the field of view of the detector. Sheath flow can be configured in numerous ways, including: The sample material is the interior material in a concentric laminar flow, with the diluent volume in the exterior; the diluent volume is on one side of the sample volume; the diluent volume is on two sides of the sample material; the diluent volume is on multiple sides of the sample material, but not enclosing the sample material completely; the diluent volume completely surrounds the sample material; the diluent volume completely surrounds the sample material concentrically; the sample material is the interior material in a discontinuous series of drops and the diluent volume completely surrounds each drop of sample material.

In some embodiments, single molecule detectors of the invention include no more than one interrogation space. In some embodiments, multiple interrogation spaces are used. Multiple interrogation spaces have been previously disclosed and are incorporated by reference from U.S. Pat. No. 7,572,640. One skilled in the art will recognize that in some cases the analyzer will contain a plurality of distinct interrogation spaces. In some embodiments, the analyzer contains 2, 3, 4, 5, 6 or more distinct interrogation spaces.

In one embodiment of the analyzer system, the particles are moved through the interrogation space by a motive force. In some embodiments, the motive force for moving particles is pressure. In some embodiments, the pressure is supplied by a pump, and air pressure source, a vacuum source, a centrifuge, or a combination thereof. In some embodiments, the motive force for moving particles is an electrokinetic force. The use of an electrokinetic force as a motive force has been previously disclosed in a prior application and is incorporated by reference from U.S. Pat. No. 7,572,640.

In one embodiment, pressure can be used as a motive force to move particles through the interrogation space of the capillary flow cell. In a further embodiment, pressure is supplied to move the sample by means of a pump. Suitable pumps are known in the art. In one embodiment, pumps manufactured for HPLC applications, such as those made by Scivax, Inc. can be used as a motive force. In other embodiments, pumps manufactured for microfluidics applications can be used when smaller volumes of sample are being pumped. Preferably all materials within the pump that come into contact with sample are made of highly inert materials, e.g., polyetheretherketone (PEEK), fused silica, or sapphire.

A motive force is necessary to move the sample through the capillary flow cell to push the sample through the interrogation space for analysis. A motive force is also required to push a flushing sample through the capillary flow cell after the sample has been passed through. A motive force is also required to push the sample back out into a sample recovery vessel, when sample recovery is employed. Standard pumps come in a variety of sizes, and the proper size may be chosen to suit the anticipated sample size and flow requirements. In some embodiments, separate pumps are used for sample analysis and for flushing of the system. The analysis pump may have a capacity of approximately 0.000001 mL to approximately 10 mL, or approximately 0.001 mL to approximately 1 mL, or approximately 0.01 mL to approximately 0.2 mL, or approximately 0.005, 0.01, 0.05, 0.1, or 0.5 mL. Flush pumps may be of larger capacity than analysis pumps. Flush pumps may have a volume of about 0.01 mL to about 20 mL, or about 0.1 mL to about 10 mL, or about 0.1 mL to about 2 mL, or about or about 0.05, 0.1, 0.5, 1, 5, or 10 mL. These pump sizes are illustrative only, and those of skill in the art will appreciate that the pump size may be chosen according to the application, sample size, viscosity of fluid to be pumped, tubing dimensions, rate of flow, temperature, and other factors well known in the art. In some embodiments, pumps of the system are driven by stepper motors, which are easy to control very accurately with a microprocessor.

In preferred embodiments, the flush and analysis pumps are used in series, with special check valves to control the direction of flow. The plumbing is designed so that when the analysis pump draws up the maximum sample, the sample does not reach the pump itself. This is accomplished by choosing the ID and length of the tubing between the analysis pump and the analysis capillary such that the tubing volume is greater than the stroke volume of the analysis pump.

In one embodiment, light (e.g., light in the ultra-violet, visible or infrared range) emitted by a fluorescent label after exposure to electromagnetic radiation is detected. The detector or detectors is capable of capturing the amplitude and duration of photon bursts from a fluorescent moiety, and further converting the amplitude and duration of the photon burst to electrical signals. Detection devices such as CCD cameras, video input module cameras, and Streak cameras can be used to produce images with contiguous signals. In another embodiment, devices such as a bolometer, a photodiode, a photodiode array, avalanche photodiodes, and photomultipliers which produce sequential signals may be used. Any combination of the aforementioned detectors may also be used. In one embodiment, avalanche photodiodes are used for detecting photons.

Using specific optics between an interrogation space and its corresponding detector, several distinct characteristics of the emitted electromagnetic radiation can be detected including: emission wavelength, emission intensity, burst size, burst duration, and fluorescence polarization. In some embodiments, the detector is a photodiode that is used in reverse bias. A photodiode set in reverse bias usually has an extremely high resistance. This resistance is reduced when light of an appropriate frequency shines on the P/N junction. Hence, a reverse biased diode can be used as a detector by monitoring the current running through it. Circuits based on this effect are more sensitive to light than ones based on zero bias.

In one embodiment of the analyzer system, the photodiode can be an avalanche photodiode, which can be operated with much higher reverse bias than conventional photodiodes, thus allowing each photo-generated carrier to be multiplied by avalanche breakdown, resulting in internal gain within the photodiode, which increases the effective responsiveness (sensitivity) of the device. The choice of photodiode is determined by the energy or emission wavelength emitted by the fluorescently labeled particle. In some embodiments, the photodiode is a silicon photodiode that detects energy in the range of 190-1100 nm; in another embodiment the photodiode is a germanium photodiode that detects energy in the range of 800-1700 nm; in another embodiment the photodiode is an indium gallium arsenide photodiode that detects energy in the range of 800-2600 nm; and in yet other embodiments, the photodiode is a lead sulfide photodiode that detects energy in the range of between less than 1000 nm to 3500 nm. In some embodiments, the avalanche photodiode is a single-photon detector designed to detect energy in the 400 nm to 1100 nm wavelength range. Single photon detectors are commercially available (e.g., Perkin Elmer, Wellesley, Mass.).

In some embodiments, the detector is an avalanche photodiode detector that detects energy between 300 nm and 1700 nm. In one embodiment, silicon avalanche photodiodes can be used to detect wavelengths between 300 nm and 1100 nm. Indium gallium arsenic photodiodes can be used to detect wavelengths between 900 nm and 1700 nm. In some embodiments, an analyzer system can include at least one detector; in other embodiments, the analyzer system can include at least two detectors, and each detector can be chosen and configured to detect light energy at a specific wavelength range. For example, two separate detectors can be used to detect particles that have been tagged with different labels, which upon excitation with an EM source, will emit photons with energy in different spectra. In one embodiment, an analyzer system can include a first detector that can detect fluorescent energy in the range of 450-700 nm such as that emitted by a green dye (e.g., Alexa Fluor 546); and a second detector that can detect fluorescent energy in the range of 620-780 nm such as that emitted by a far-red dye (e.g., ALEXA FLUOR® 647). Detectors for detecting fluorescent energy in the range of 400-600 nm such as that emitted by blue dyes (e.g., Hoechst 33342), and for detecting energy in the range of 560-700 nm such as that emitted by red dyes (ALEXA FLUOR® and Cy3) can also be used.

A system comprising two or more detectors can be used to detect individual particles that are each tagged with two or more labels that emit light in different spectra. For example, two different detectors can detect an antibody that has been tagged with two different dye labels. Alternatively, an analyzer system comprising two detectors can be used to detect particles of different types, each type being tagged with different dye molecules, or with a mixture of two or more dye molecules. For example, two different detectors can be used to detect two different types of antibodies that recognize two different proteins, each type being tagged with a different dye label or with a mixture of two or more dye label molecules. By varying the proportion of the two or more dye label molecules, two or more different particle types can be individually detected using two detectors. It is understood that three or more detectors can be used without departing from the scope of the invention.

It should be understood by one skilled in the art that one or more detectors can be configured at each interrogation space, whether one or more interrogation spaces are defined within a flow cell, and that each detector may be configured to detect any of the characteristics of the emitted electromagnetic radiation listed above. The use of multiple detectors, e.g., for multiple interrogation spaces, has been previously disclosed in a prior application and is incorporated by reference here from U.S. Pat. No. 7,572,640. Once a particle is labeled to render it detectable (or if the particle possesses an intrinsic characteristic rendering it detectable), any suitable detection mechanism known in the art may be used without departing from the scope of the present invention, for example a CCD camera, a video input module camera, a Streak camera, a bolometer, a photodiode, a photodiode array, avalanche photodiodes, and photomultipliers producing sequential signals, and combinations thereof. Different characteristics of the electromagnetic radiation may be detected including: emission wavelength, emission intensity, burst size, burst duration, fluorescence polarization, and any combination thereof.

In a further embodiment, the analyzer system may include a sampling system to prepare the sample for introduction into the analyzer system. The sampling system included is capable of automatically sampling a plurality of samples and providing a fluid communication between a sample container and a first interrogation space.

In some embodiments, the analyzer system of the invention includes a sampling system for introducing an aliquot of a sample into the single particle analyzer for analysis. Any mechanism that can introduce a sample may be used. Samples can be drawn up using either a vacuum suction created by a pump or by pressure applied to the sample that would push liquid into the tube, or by any other mechanism that serves to introduce the sample into the sampling tube. Generally, but not necessarily, the sampling system introduces a sample of known sample volume into the single particle analyzer; in some embodiments where the presence or absence of a particle or particles is detected, precise knowledge of the sample size is not critical. In preferred embodiments the sampling system provides automated sampling for a single sample or a plurality of samples. In embodiments where a sample of known volume is introduced into the system, the sampling system provides a sample for analysis of more than about 0.0001, 0.001, 0.01, 0.1, 1, 2, 5, 10, 20, 30, 40, 50, 60, 70, 80, 90, 100, 150, 200, 500, 1000, 1500, or 2000 μl.

Because of the sensitivity of the methods of the present invention, very small sample volumes can be used. For example, 10 μl or less.

In some embodiments, the sampling system provides a sample size that can be varied from sample to sample. In these embodiments, the sample size may be any one of the sample sizes described herein, and may be changed with every sample, or with sets of samples, as desired.

Sample volume accuracy, and sample to sample volume precision of the sampling system, is required for the analysis at hand. In some embodiments, the precision of the sampling volume is determined by the pumps used, typically represented by a CV of less than about 50, 40, 30, 20, 10, 5, 4, 3, 2, 1, 0.5, 0.1, 0.05, or 0.01% of sample volume. In some embodiments, the sample to sample precision of the sampling system is represented by a CV of less than about 50, 40, 30, 20, 10, 5, 4, 3, 2, 1, 0.5, 0.1, 0.05, or 0.01%. In some embodiments, the intra-assay precision of the sampling system is represented by a CV of less than about 10, 5, 1, 0.5, or 0.1%.

In some embodiments, the sampling system provides low sample carryover, advantageous in that an additional wash step is not required between samples. Thus, in some embodiments, sample carryover is less than about 1, 0.5, 0.1, 0.05, 0.04, 0.03, 0.02, 0.01, 0.005, or 0.001%. In some embodiments, sample carryover is less than about 0.02%. In some embodiments, sample carryover is less than about 0.01%.

In some embodiments the sampler provides a sample loop. In these embodiments, multiple samples are drawn into tubing sequentially and each is separated from the others by a “plug” of buffer. The samples typically are read one after the other with no flushing in between. Flushing is done once at the end of the loop. In embodiments where a buffer “plug” is used, the plug may be recovered ejecting the buffer plug into a separate well of a microtiter plate.

The sampling system may be adapted for use with standard assay equipment, for example, a 96-well microtiter plate, or, preferably, a 384-well plate. In some embodiments the system includes a 96 well plate positioner and a mechanism to dip the sample tube into and out of the wells, e.g., a mechanism providing movement along the X, Y, and Z axes. In some embodiments, the sampling system provides multiple sampling tubes from which samples may be stored and extracted from, when testing is commenced. In some embodiments, all samples from the multiple tubes are analyzed on one detector. In other embodiments, multiple single molecule detectors may be connected to the sample tubes. Samples may be prepared by steps that include operations performed on sample in the wells of the plate prior to sampling by the sampling system, or sample may be prepared within the analyzer system, or some combination of both.

Sample preparation includes the steps necessary to prepare a raw sample for analysis. These steps can involve, by way of example, one or more steps of: separation steps such as centrifugation, filtration, distillation, chromatography; concentration, cell lysis, alteration of pH, addition of buffer, addition of diluents, addition of reagents, heating or cooling, addition of label, binding of label, cross-linking with illumination, separation of unbound label, inactivation and/or removal of interfering compounds and any other steps necessary for the sample to be prepared for analysis by the single particle analyzer. In some embodiments, blood is treated to separate out plasma or serum. Additional labeling, removal of unbound label, and/or dilution steps may also be performed on the serum or plasma sample.

In some embodiments, the analyzer system includes a sample preparation system that performs some or all of the processes needed to provide a sample ready for analysis by the single particle analyzer. This system may perform any or all of the steps listed above for sample preparation. In some embodiments samples are partially processed by the sample preparation system of the analyzer system. Thus, in some embodiments, a sample may be partially processed outside the analyzer system first. For example, the sample may be centrifuged first. The sample may then be partially processed inside the analyzer by a sample preparation system. Processing inside the analyzer includes labeling the sample, mixing the sample with a buffer and other processing steps that will be known to one in the art. In some embodiments, a blood sample is processed outside the analyzer system to provide a serum or plasma sample, which is introduced into the analyzer system and further processed by a sample preparation system to label the particle or particles of interest and, optionally, to remove unbound label. In other embodiments preparation of the sample can include immunodepletion of the sample to remove particles that are not of interest or to remove particles that can interfere with sample analysis. In yet other embodiments, the sample can be depleted of particles that can interfere with the analysis of the sample. For example, sample preparation can include the depletion of heterophilic antibodies, which are known to interfere with immunoassays that use non-human antibodies to directly or indirectly detect a particle of interest. Similarly, other proteins that interfere with measurements of the particles of interest can be removed from the sample using antibodies that recognize the interfering proteins.

In some embodiments, the sample can be subjected to solid phase extraction prior to being assayed and analyzed. Solid phase extraction can be used to remove the basic matrix of a sample, which can diminish the sensitivity of the assay. In yet other embodiments, the particles of interest present in a sample may be concentrated by drying or lyophilizing a sample and solubilizing the particles in a smaller volume than that of the original sample.

In some embodiments the analyzer system provides a sample preparation system that provides complete preparation of the sample to be analyzed on the system, such as complete preparation of a blood sample, a saliva sample, a urine sample, a cerebrospinal fluid sample, a lymph sample, a biopsy sample, and the like. In some embodiments the analyzer system provides a sample preparation system that provides some or all of the sample preparation. In some embodiments, the initial sample is a blood sample that is further processed by the analyzer system. In some embodiments, the sample is a serum or plasma sample that is further processed by the analyzer system. The serum or plasma sample may be further processed by, e.g., contacting with a label that binds to a particle or particles of interest; the sample may then be used with or without removal of unbound label.

In some embodiments, sample preparation is performed, either outside the analysis system or in the sample preparation component of the analysis system, on one or more microtiter plates, such as a 96-well plate. Reservoirs of reagents, buffers, and the like can be in intermittent fluid communication with the wells of the plate by means of tubing or other appropriate structures, as are well-known in the art. Samples may be prepared separately in 96 well plates or tubes. Sample isolation, label binding and, if necessary, label separation steps may be done on one plate. In some embodiments, prepared particles are then released from the plate and samples are moved into tubes for sampling into the sample analysis system. In some embodiments, all steps of the preparation of the sample are done on one plate and the analysis system acquires sample directly from the plate. Although this embodiment is described in terms of a 96-well plate, it will be appreciated that any vessel for containing one or more samples and suitable for preparation of sample may be used. For example, standard microtiter plates of 384 or 1536 wells may be used.

Microfluidics systems may also be used for sample preparation and as sample preparation systems that are part of analyzer systems, especially for samples suspected of containing concentrations of particles high enough that detection requires smaller samples. Samples may be prepared in series or in parallel, for use in a single or multiple analyzer systems.

In some embodiments, the sample includes a buffer. The buffer may be mixed with the sample outside the analyzer system, or it may be provided by the sample preparation mechanism. While any suitable buffer can be used, the preferable buffer has low fluorescence background, is inert to the detectably labeled particle, can maintain the working pH and, in embodiments wherein the motive force is electrokinetic, has suitable ionic strength for electrophoresis. The buffer concentration can be any suitable concentration, such as in the range from about 1 to about 200 mM. Any buffer system may be used as long as it provides for solubility, function, and delectability of the molecules of interest. In some embodiments, e.g., for application using pumping, the buffer is selected from the group consisting of phosphate, glycine, acetate, citrate, acidulate, carbonate/bicarbonate, imidazole, triethanolamine, glycine amide, borate, MES, Bis-Tris, ADA, aces, PIPES, MOPSO, Bis-Tris Propane, BES, MOPS, TES, HEPES, DIPSO, MOBS, TAPSO, Trizma, HEPPSO, POPSO, TEA, EPPS, Tricine, Gly-Gly, Bicine, HEPBS, TAPS, AMPD, TABS, AMPSO, CHES, CAPSO, AMP, CAPS, and CABS. The buffer can also be selected from the group consisting of Gly-Gly, bicine, tricine, 2-morpholine ethanesulfonic acid (MES), 4-morpholine propanesulfonic acid (MOPS) and 2-amino-2-methyl-1-propanol hydrochloride (AMP). A useful buffer is 2 mM Tris/borate at pH 8.1, but Tris/glycine and Tris/HCl are also acceptable. Other buffers are as described herein.

Buffers useful for electrophoresis are disclosed in a prior application and are incorporated by reference herein from U.S. Pat. No. 7,572,640.

One highly useful feature of embodiments of the analyzers and analysis systems of the invention is that the sample can be analyzed without consuming it. This can be especially important when sample materials are limited. Recovering the sample also allows one to do other analyses or reanalyze it. The advantages of this feature for applications where sample size is limited and/or where the ability to reanalyze the sample is desirable, e.g., forensic, drug screening, and clinical diagnostic applications, will be apparent to those of skill in the art.

Thus, in some embodiments, the analyzer system of the invention further provides a sample recovery system for sample recovery after analysis. In these embodiments, the system includes mechanisms and methods by which the sample is drawn into the analyzer, analyzed and then returned, e.g., by the same path, to the sample holder, e.g., the sample tube. Because no sample is destroyed and because it does not enter any of the valves or other tubing, it remains uncontaminated. In addition, because all the materials in the sample path are highly inert, e.g., PEEK, fused silica, or sapphire, there is little contamination from the sample path. The use of the stepper motor controlled pumps (particularly the analysis pump) allows precise control of the volumes drawn up and pushed back out. This allows complete or nearly complete recovery of the sample with little if any dilution by the flush buffer. Thus, in some embodiments, more than about 50%, 60%, 70%, 80%, 85%, 90%, 95%, 96%, 97%, 98%, 99%, 99.5%, or 99.9% of the sample is recovered after analysis. In some embodiments, the recovered sample is undiluted. In some embodiments, the recovered sample is diluted less than about 1.5-fold, 1.4-fold, 1.3-fold, 1.2-fold, 1.1-fold, 1.05-fold, 1.01-fold, 1.005-fold, or 1.001-fold.

For sampling and/or sample recovery, any mechanism for transporting a liquid sample from a sample vessel to the analyzer may be used. In some embodiments the inlet end of the analysis capillary has attached a short length of tubing, e.g., PEEK tubing that can be dipped into a sample container, e.g., a test tube or sample well, or can be held above a waste container. When flushing, to clean the previous sample from the apparatus, this tube is positioned above the waste container to catch the flush waste. When drawing a sample in, the tube is put into the sample well or test tube. Typically the sample is drawn in quickly, and then pushed out slowly while observing particles within the sample. Alternatively, in some embodiments, the sample is drawn in slowly during at least part of the draw-in cycle; the sample may be analyzed while being slowly drawn in. This can be followed by a quick return of the sample and a quick flush. In some embodiments, the sample may be analyzed both on the inward (draw-in) and outward (pull out) cycle, which improves counting statistics, e.g., of small and dilute samples, as well as confirming results, and the like. If it is desired to save the sample, it can be pushed back out into the same sample well it came from, or to another. If saving the sample is not desired, the tubing is positioned over the waste container.

### EXAMPLES

### Example 1

### Alzheimer's Disease Biomarkers

36 potential biomarkers for Alzheimer's disease (AD) were analyzed both individually and in combination for their ability to (1) aid in diagnosing AD; (2) differentiate AD form healthy subjects; or (3) predict and/or rank the severity of AD using cerebral spinal fluid (CSF) samples from patients. In order to determine which biomarker or combinations of biomarkers best diagnose AD and/or best predict AD severity, CSF samples were acquired from a commercial vendor. The CSF samples included samples from the following cohorts: 16 AD patients ranging from 62-87 years of age and with a median mini-mental state examination (MMSE) of 17; 5 Parkinson's Disease (PD) patients ranging from 63-74 years of age and with a median MMSE of 29; and 10 healthy control patients ranging in age from 60-77 years and with a median MMSE of 29.

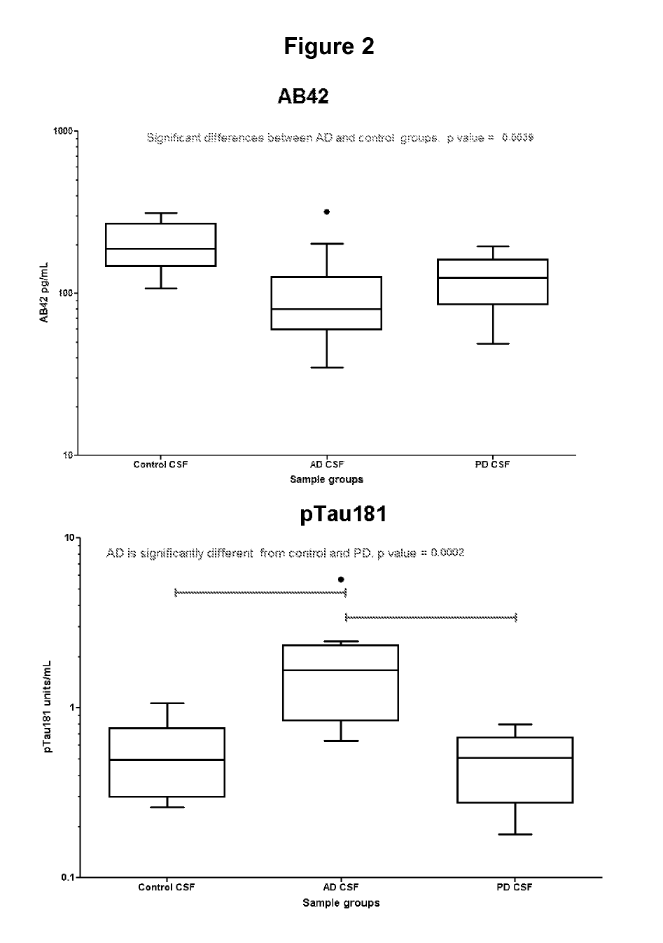

The 36 biomarkers tested are listed in the table of FIG. 1. Of the 36 biomarkers tested, many were determined to be non-measurable in previously published studies. In order to validate the CSF samples, known AD biomarkers Abeta-42 and pTau-181 were among the 36 biomarkers tested. Tukey box plots were generated for each biomarker and the Kruskal-Wallis test was used to determine statistical significance. As expected, Abeta-42 levels were decreased and pTau-181 levels were increased in the AD CSF samples (FIG. 2); thus validating the cohort.

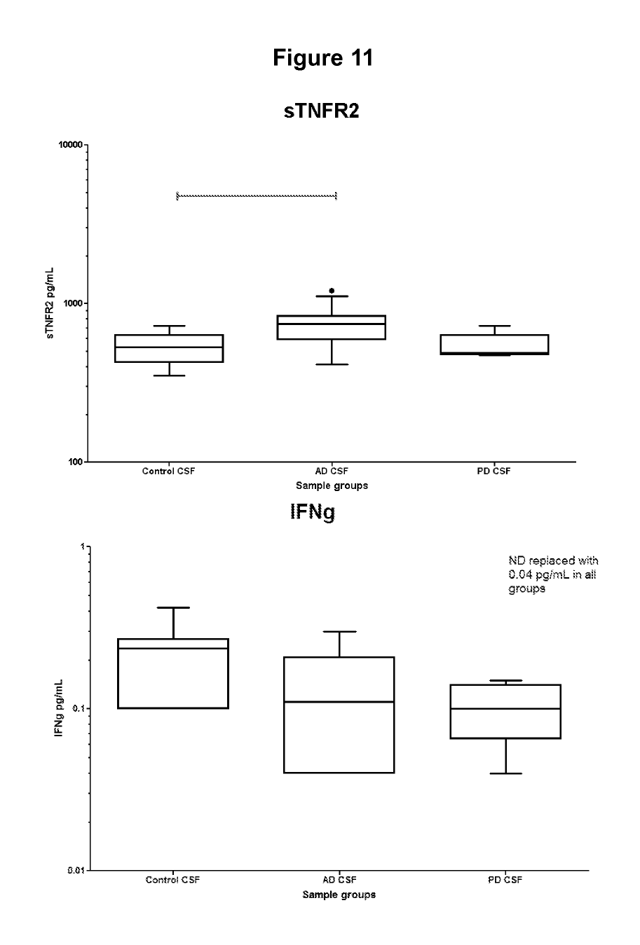

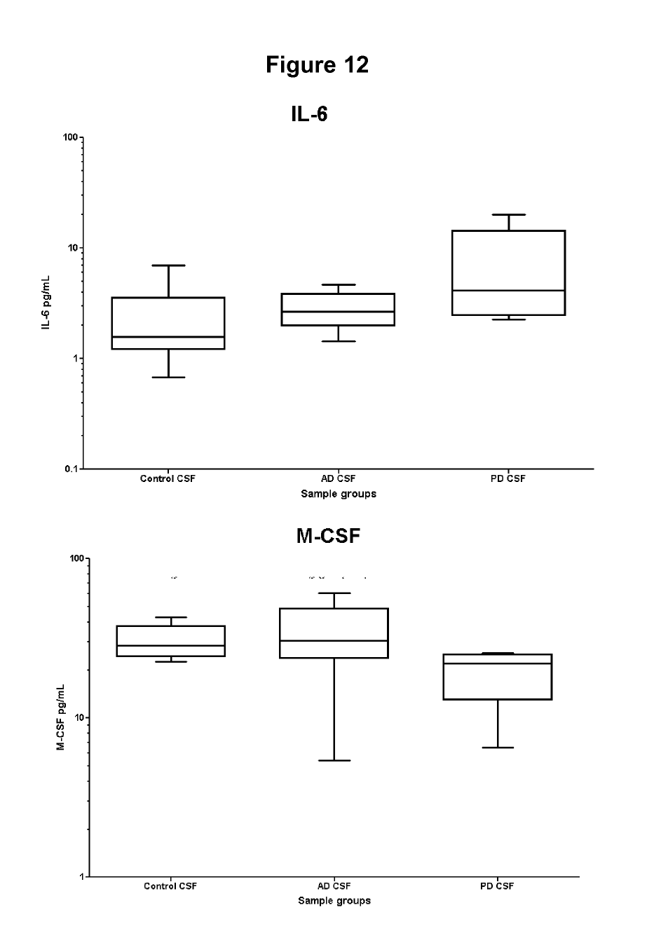

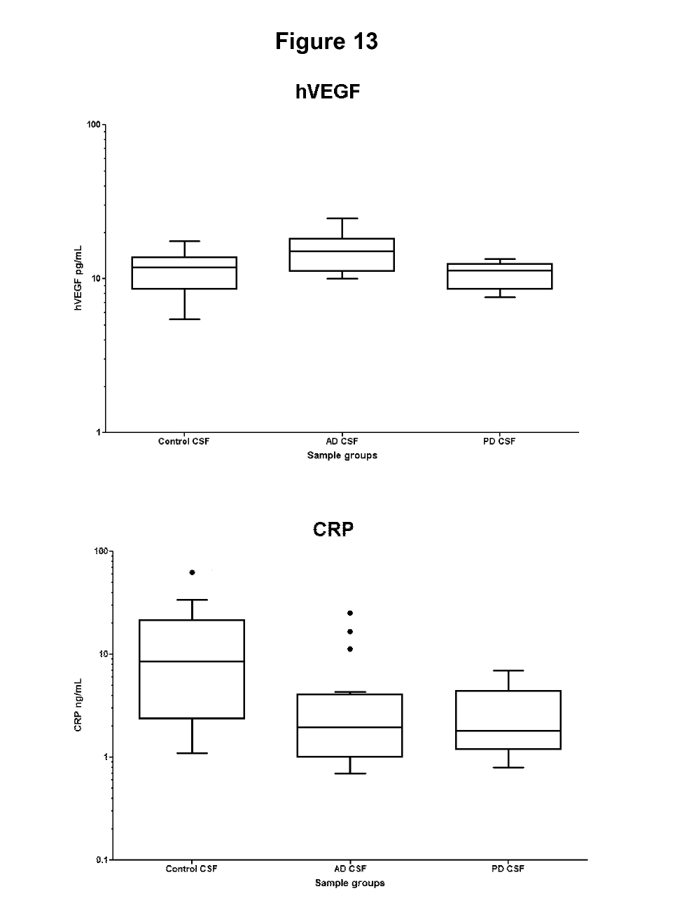

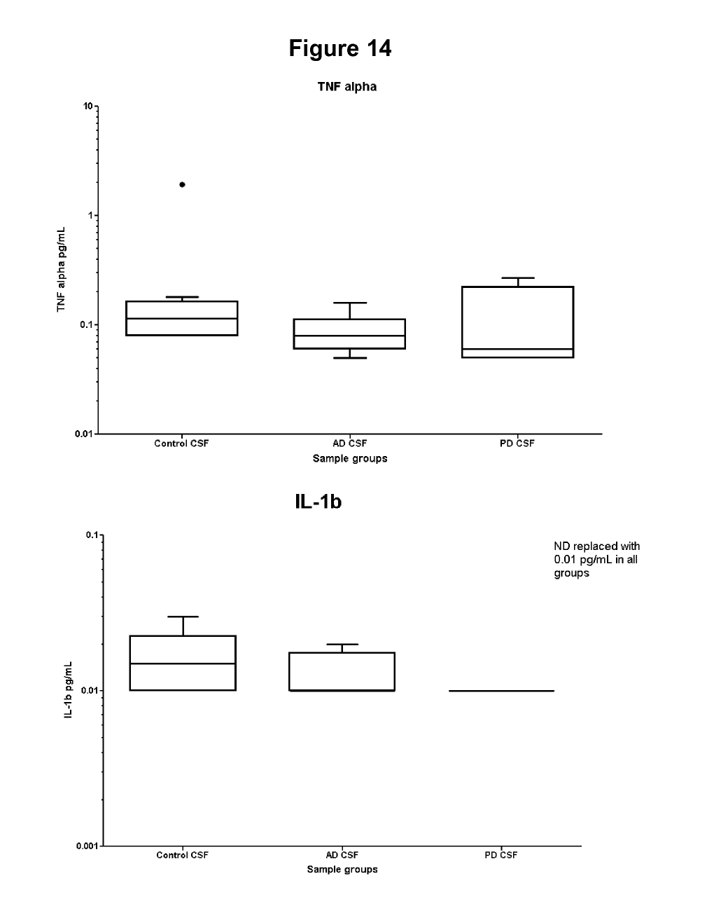

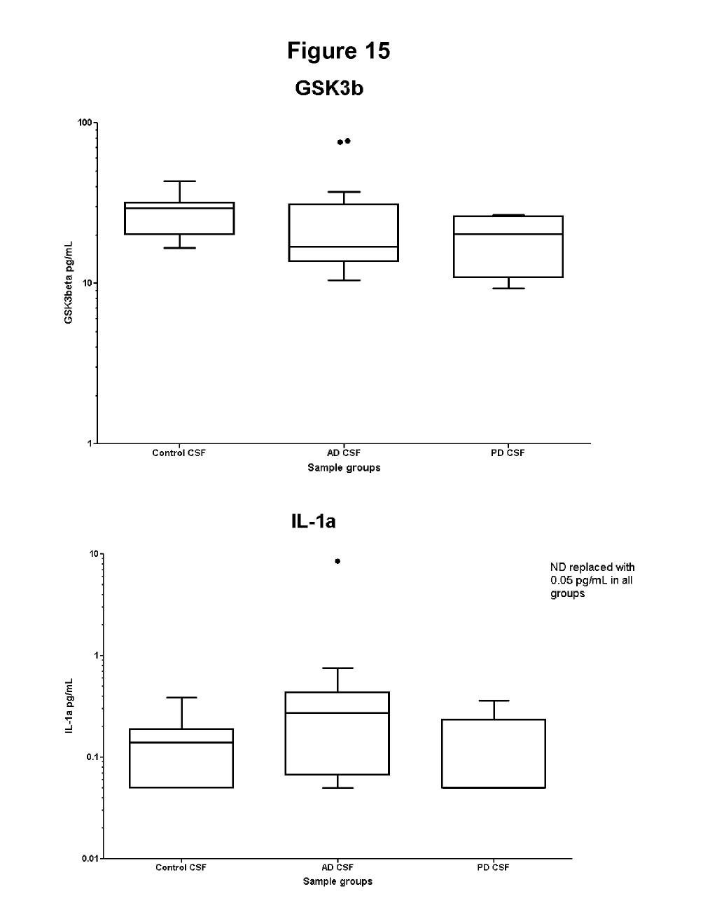

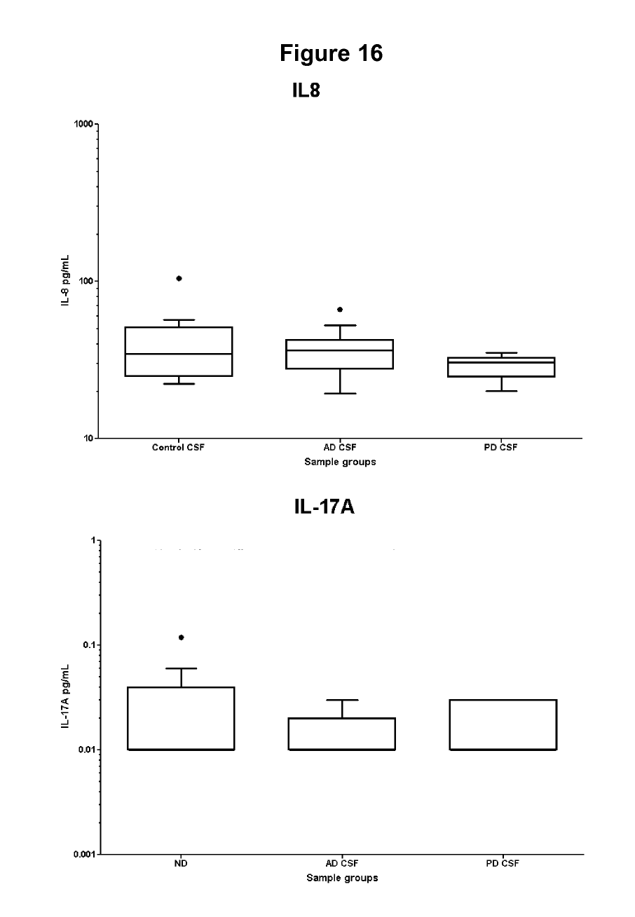

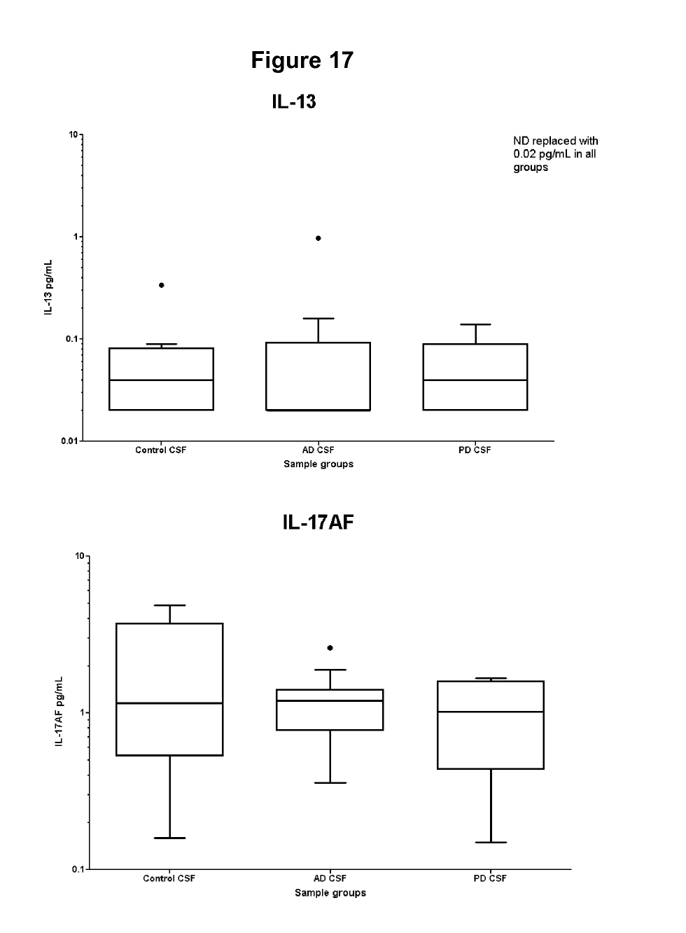

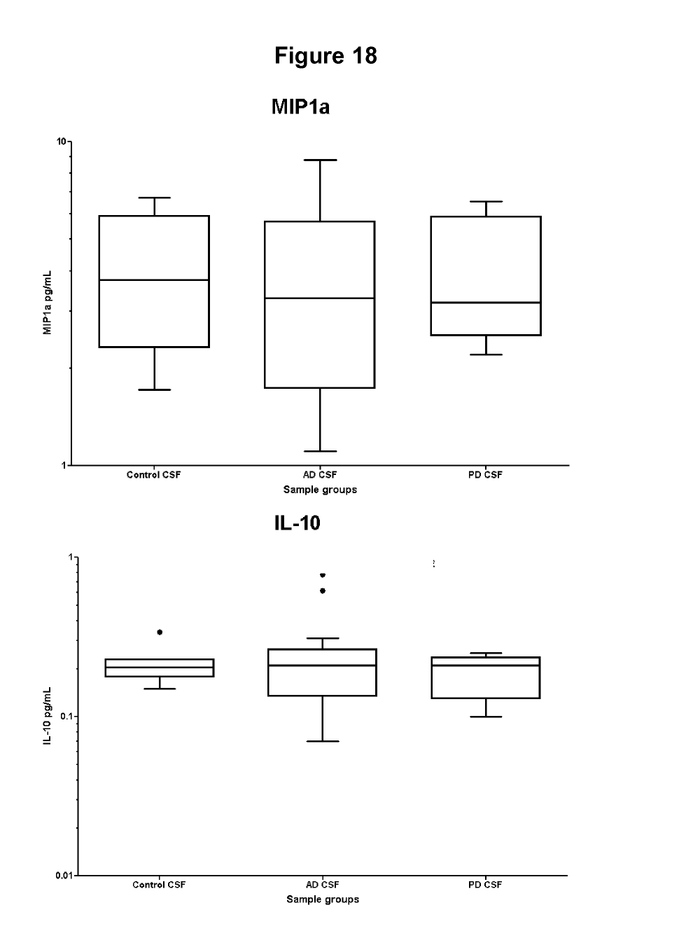

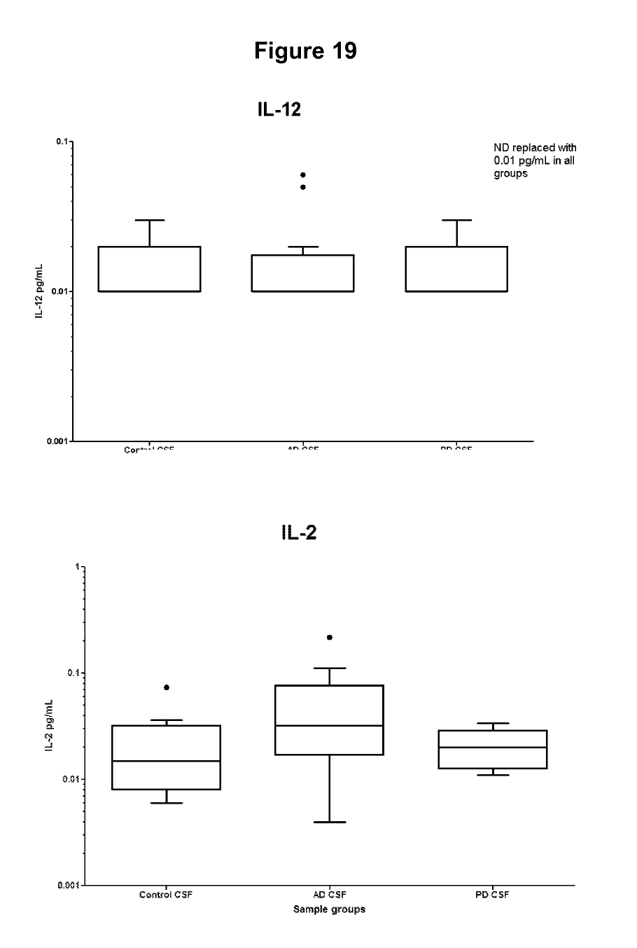

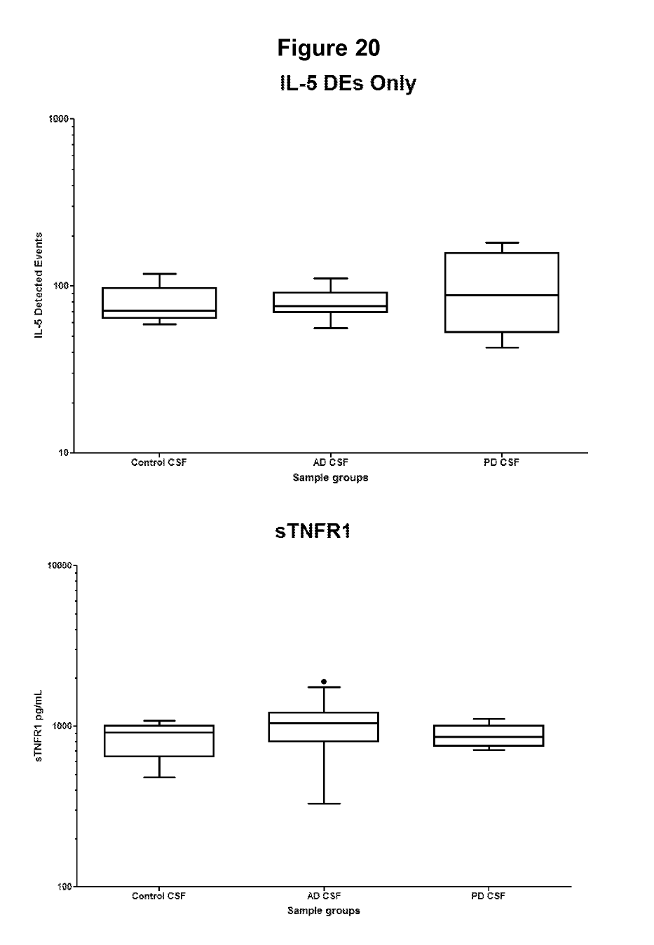

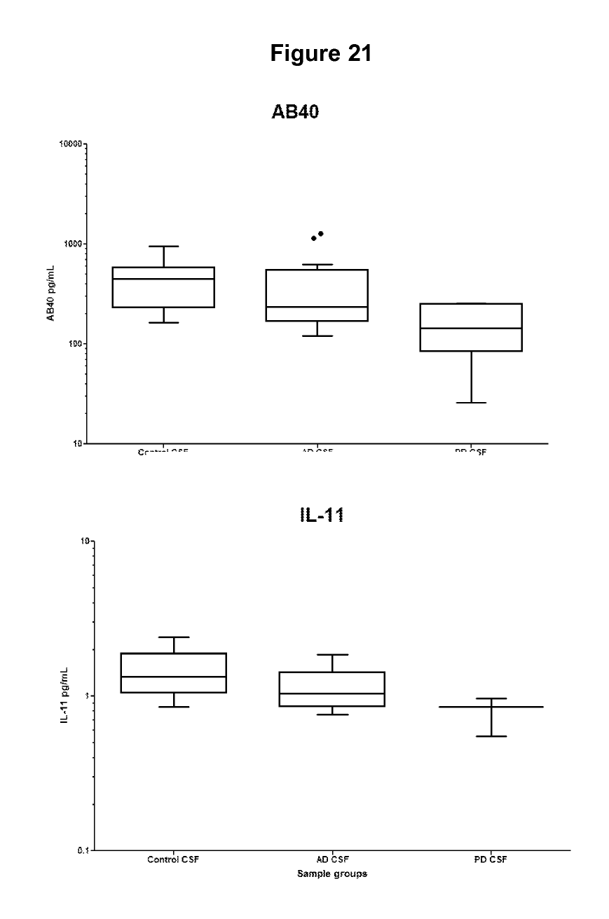

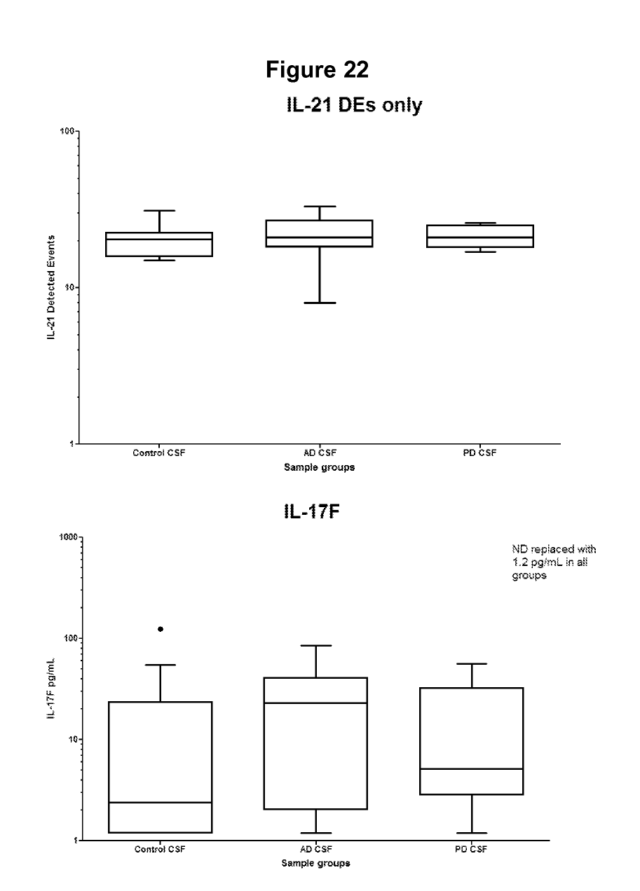

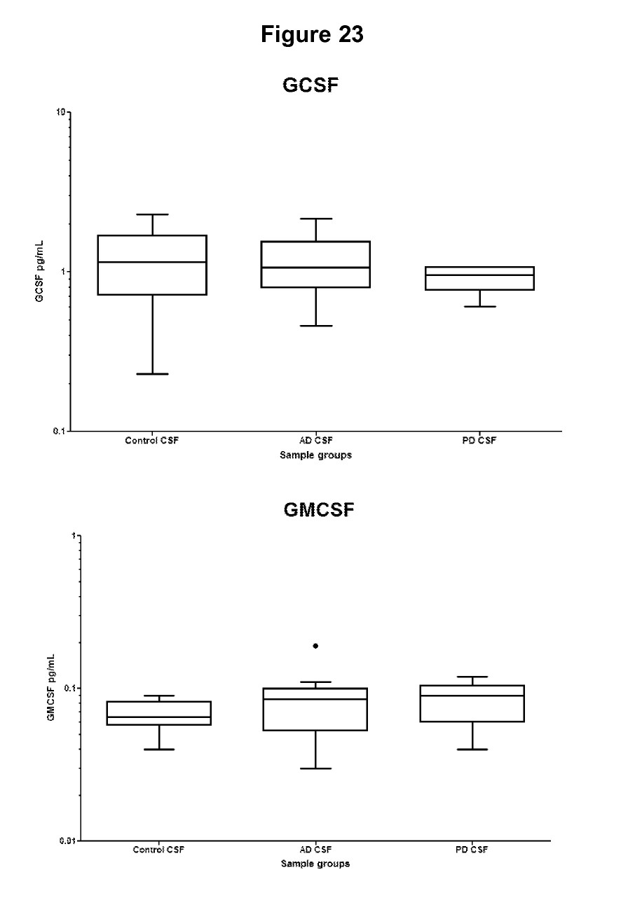

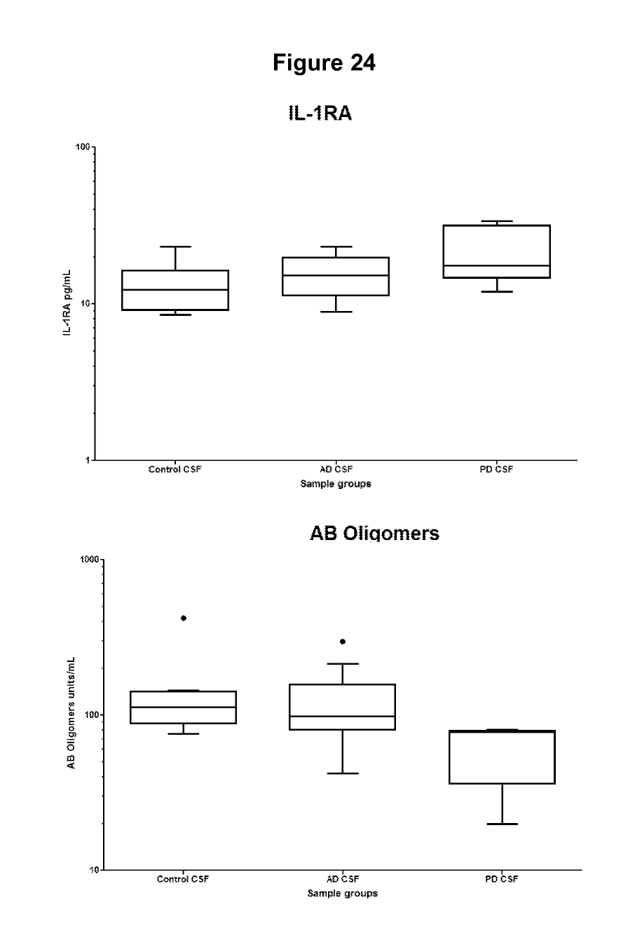

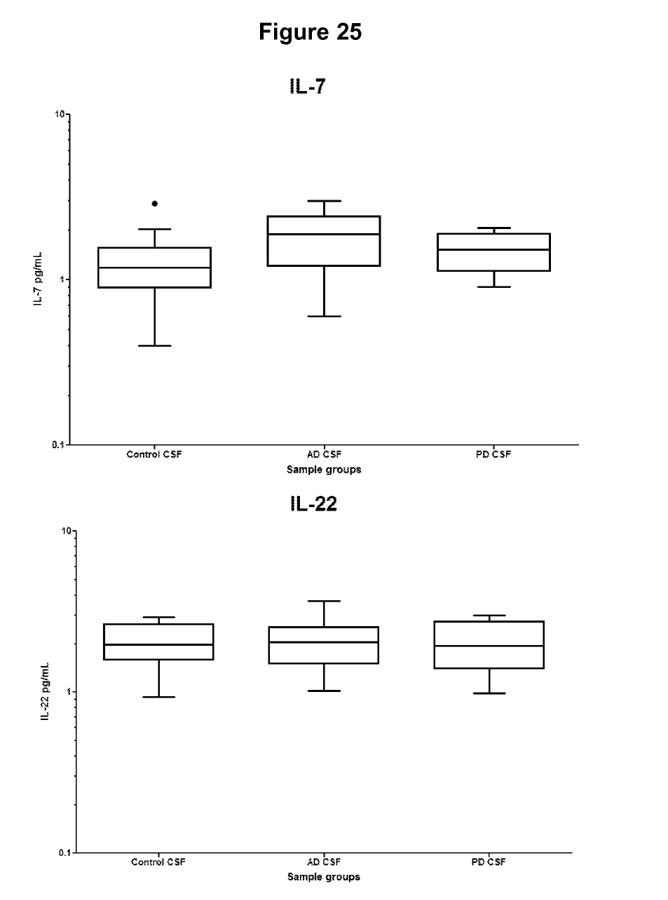

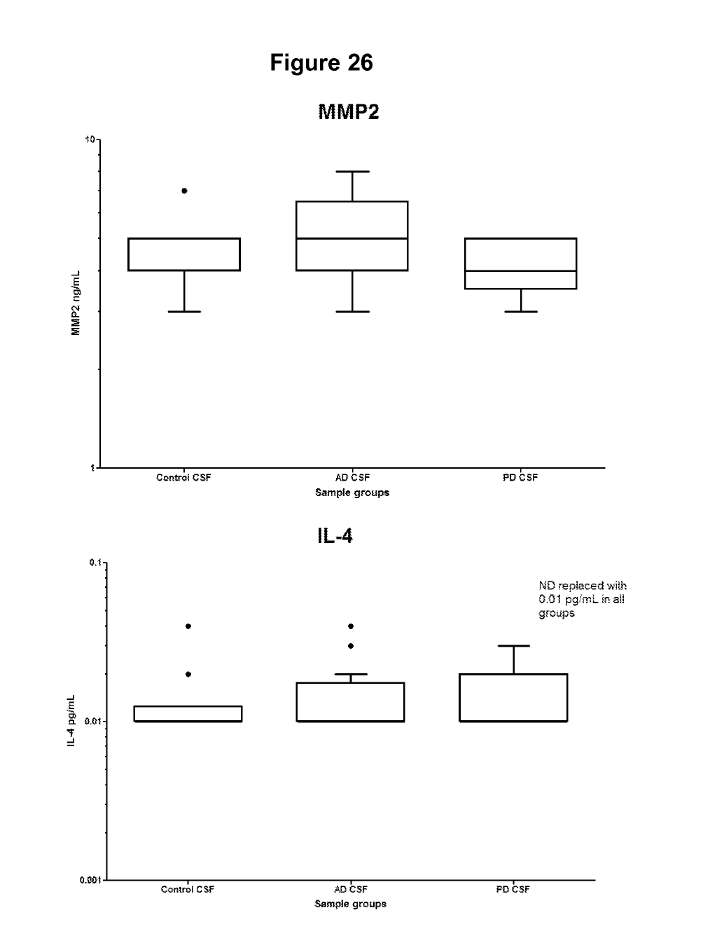

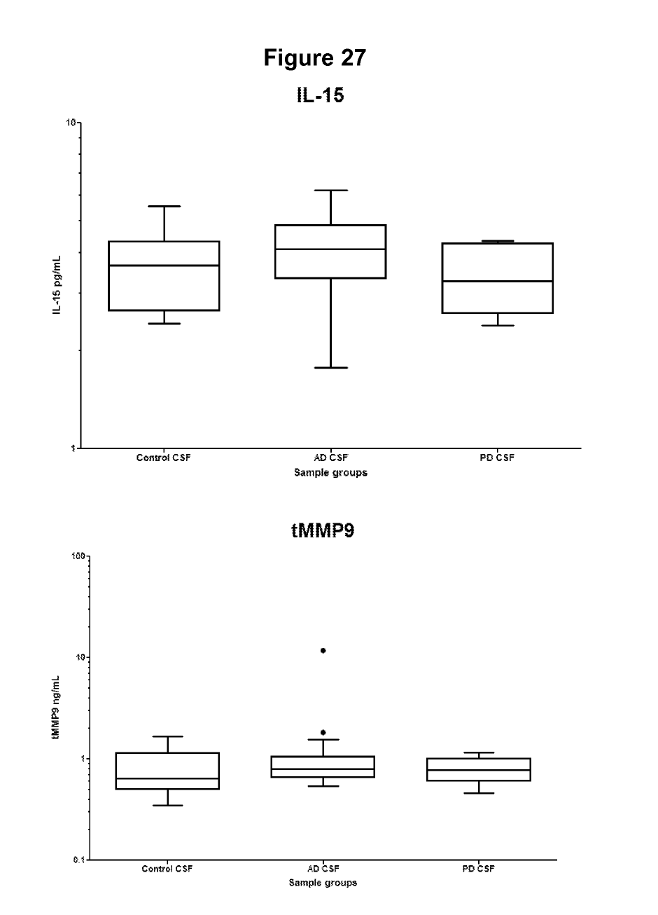

In order to determine if there were differences between AD and control samples for each of the 36 biomarkers, each of the biomarkers was tested in the CSF samples. Tukey box plots were generated for each biomarker and the Kruskal-Wallis test was used to determine statistical significance, P-values, comparing 3 or more unpaired groups with non-Gaussian distributions (results of each biomarker alone are shown in and FIGS. 1, 2, and 11-28). The horizontal bar in each group represents the mean, the box represents 50% of data, the vertical bars represent 95% of the data, and outliers that are 3 standard deviations or more of the mean are shown as individual dots.

In addition to studying each of the biomarkers individually, biomarkers were analyzed in combinations using logistic regression and general linear models of Log₁₀ transformed data. In order to identify which biomarkers to include in a predictive algorithm Spearman correlation with MMSE was used. Analyses were performed: 1) comparing AD vs. controls (including healthy controls and PD patients) (see Example 2); and 2) comparing AD vs. healthy controls without PD patients (see Example 3).

### Example 2

### Analysis of AD Patients vs. Control (Including PD Patients)

In order to identify which biomarkers to include in a predictive algorithm, the Spearman correlation with MMSE was used. The results are shown in Table 1 below. Only biomarkers with correlations of r>0.4 are shown. Abeta-42 and pTau-181 further validate the cohort, and IL-2 surprisingly demonstrated predictive value as a biomarker for AD.

TABLE 1
Matrix of Spearman correlations with MMSE for biomarkers shown

| | MMSE | Abeta-42 | TNFa | pTau181 | CRP | IFNg | sTNFR2 | IL-2 |
|---|---|---|---|---|---|---|---|---|
| MMSE | 1 | 0.61 | 0.32 | −0.62 | 0.31 | 0.31 | −0.33 | −0.47 |
| Abeta-42 | | 1 | 0.35 | −0.26 | 0.34 | 0.41 | −0.14 | −0.2 |
| TNFa | | | 1 | −0.12 | 0.07 | 0.42 | 0.15 | −0.05 |
| pTau181 | | | | 1 | −0.16 | −0.2 | 0.61 | 0.2 |
| CRP | | | | | 1 | 0.32 | −0.02 | −0.38 |
| IFNg | | | | | | 1 | 0.05 | −0.21 |
| sTNFR2 | | | | | | | 1 | 0.04 |
| IL-2 | | | | | | | | 1 |

In order to determine if single biomarkers could be used to predict AD (AD with MMSE score<26) logistic regression analysis was used. All biomarkers were tested in Log-transformed models and the results are shown in Table 2 for biomarkers with area under ROC curve of 0.65. The results for Abeta-42 and pTau-181 further validate the cohort, and surprisingly the biomarker sTNFR2 (listed as sTNF-II in FIG. 1) was identified as a biomarker that is competitive with known biomarkers Abeta-42 and pTau-181.

TABLE 2
Area under ROC curve for each individual biomarker shown

| Biomarker | AuROC |
|---|---|
| pTau-181 | 0.93 |
| sTNFR2 | 0.82 |
| Abeta-42 | 0.80 |
| TNF-alpha | 0.67 |
| IL-2 | 0.66 |
| IFNg | 0.66 |
| CRP | 0.65 |

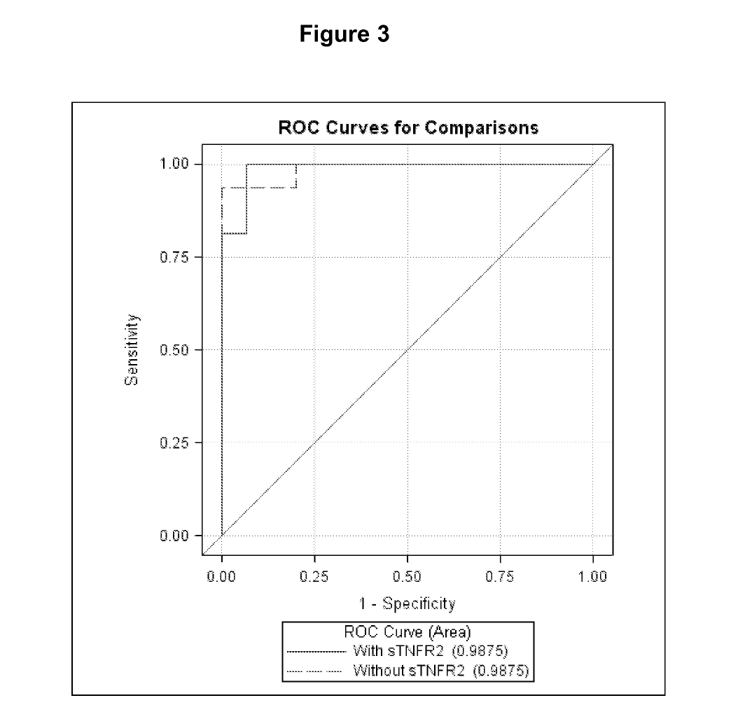

FIG. 3 shows there results of a logistic regression analysis that was used to determine if a combination of Abeta-42, pTau-181 and sTNFR2 biomarkers could predict AD. The results for the combination of Abeta-42 and pTau-181 (AuROC=0.9875) further validate the cohort (leaving little room for improvement). Adding the biomarker sTNFR2 to the combination of Abeta-42 and pTau-181 demonstrate sTNFR2 does not decrease the prediction (AuROC=0.9875). In this analysis, the non-Alzheimer's group included Parkinson's disease patients.

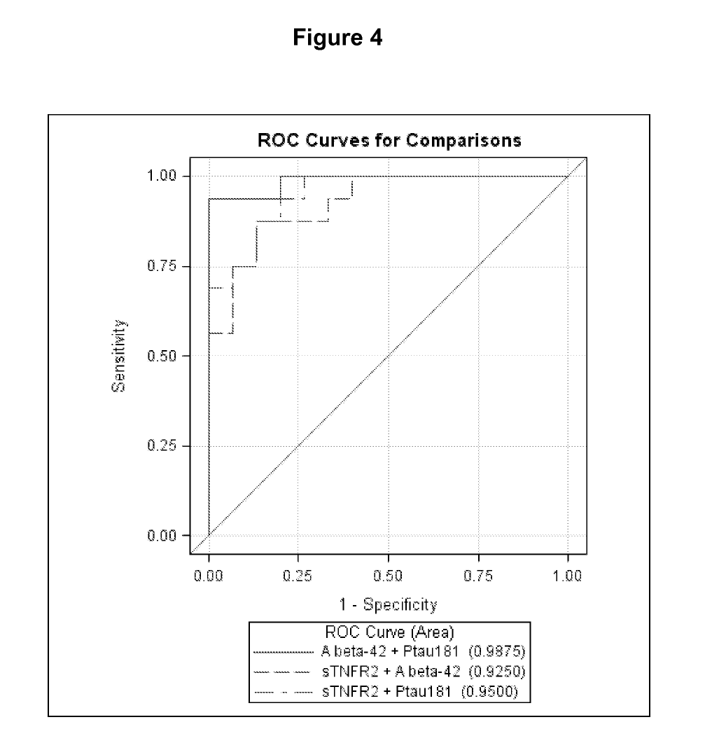

FIG. 4 shows the results of logistic regression analysis to determine if a combination of Abeta-42 with either pTau-181 or sTNFR2 biomarkers could be used to predict AD. The results for the combination of Abeta-42 and pTau-181 (AuROC=0.9875) further validate the cohort. The results for the combination of Abeta-42 and sTNFR2 provide a strong correlation (AuROC=0.9250); and results for the combination of pTau-181 and sTNFR2 also demonstrate a strong correlation (AuROC=0.9500). In this analysis, the non-Alzheimer's group included Parkinson's disease patients.

### Example 3

### Analysis of AD Patients Vs. Control (Excluding PD Patients)

In order to identify which biomarkers to include in a predictive algorithm, a Spearman correlation with MMSE was used (patients with PD were excluded from this part of the study). The results are shown in Table 3 below. Abeta-42 and pTau-181 again validate the cohort; and CRP and IL-2 were surprisingly demonstrated predictive value as biomarkers for AD.

TABLE 3
Matrix of Spearman correlations with MMSE for biomarkers shown

| | | A beta- | Ptau- | | | | | | | AB |
|---|---|---|---|---|---|---|---|---|---|---|
| | MMSE | 42 | 181 | CRP | IL-6 | IFNg | sTNFR2 | IL-2 | AB40 | Oligomers |
| MMSE | 1 | 0.63 | −0.59 | 0.46 | −0.37 | 0.39 | −0.32 | −0.54 | 0.36 | 0.39 |
| A beta-42 | | 1 | −0.34 | 0.44 | −0.03 | 0.44 | −0.23 | −0.27 | 0.69 | 0.53 |
| Ptau-181 | | | 1 | −0.27 | 0.44 | −0.36 | 0.61 | 0.19 | 0.16 | 0.21 |
| CRP | | | | 1 | 0.16 | 0.29 | −0.02 | −0.39 | 0.31 | 0.4 |
| IL-6 | | | | | 1 | 0.1 | 0.34 | −0.01 | 0.12 | 0.01 |
| IFNg | | | | | | 1 | 0 | −0.24 | 0.17 | 0.13 |
| sTNFR2 | | | | | | | 1 | −0.02 | 0.3 | 0.38 |
| IL-2 | | | | | | | | 1 | −0.04 | −0.1 |
| AB40 | | | | | | | | | 1 | 0.8 |
| AB Oligomers | | | | | | | | | | 1 |

Logistic regression analysis was used to determine if single biomarkers could be used to predict AD (AD with MMSE score<26). All biomarkers were tested in Log-transformed models and the results are shown in Table 4 for biomarkers with area under ROC curve of 0.58. The results for Abeta-42 and pTau-181 validate the cohort, and surprisingly the biomarker sTNFR2 was identified as a biomarker that is competitive with known biomarkers Abeta-42 and pTau-181 (see Table 4), and IFNg and CRP also demonstrate strong correlations.

TABLE 4
Area under ROC curve for each individual biomarker shown

| Biomarker | AuROC |
|---|---|
| pTau | 0.92 |
| Abeta-42 | 0.88 |
| sTNFR2 | 0.83 |
| IFNg | 0.75 |
| CRP | 0.74 |
| IL-6 | 0.71 |
| IL-2 | 0.67 |
| AB-40 | 0.92 |
| AB-oligomers | 0.58 |

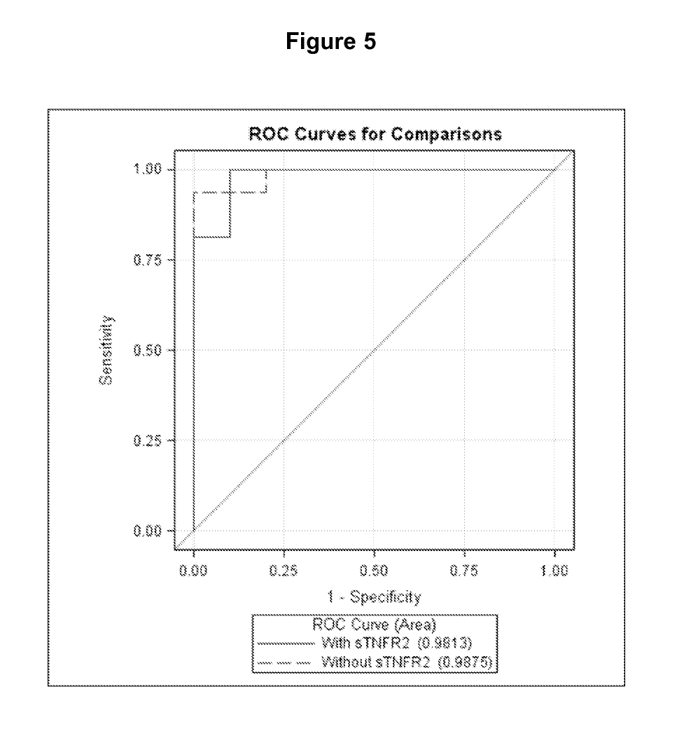

Logistic regression analysis was used to determine if a combination of Abeta-42, pTau-181 and sTNFR2 biomarkers could predict AD. The results for the combination of Abeta-42 and pTau-181 (AuROC=0.9875) further validate the cohort, and adding the biomarker sTNFR2 to Abeta-42 and pTau-181 demonstrate sTNFR2 does not improve the prediction (AuROC=0.9813) (see FIG. 5) when combined with known biomarkers Abeta-42 and pTau-181. In this analysis, the non-Alzheimer's group did not include Parkinson's disease patients.

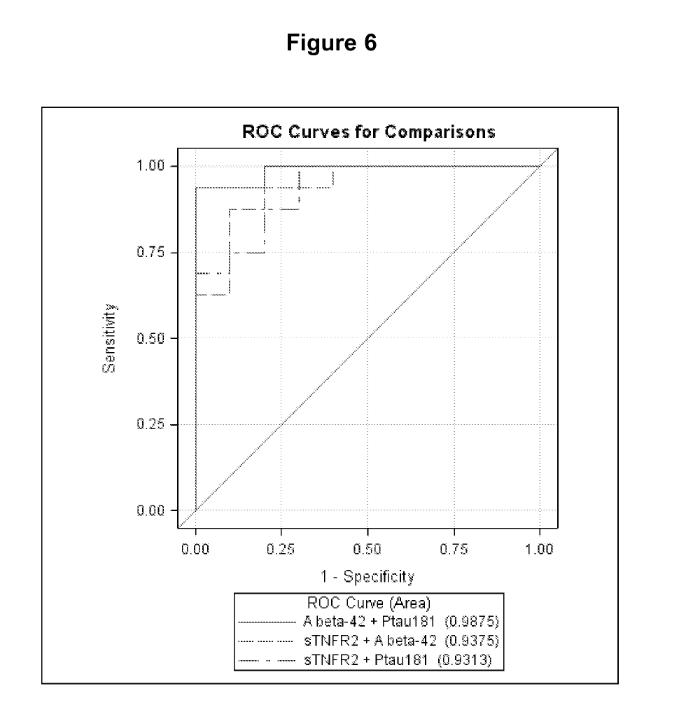

Logistic regression analysis was used to determine if a combination of Abeta-42 in combination with either pTau-181 or sTNFR2 biomarkers could predict AD. The results for the combination of Abeta-42 and pTau-181 (AuROC=0.9875) validate the cohort. The results for the combination of Abeta-42 and sTNFR2 provide a strong correlation (AuROC=0.9375); and results for the combination of pTau-181 and sTNFR2 also demonstrate a strong correlation (AuROC=0.9313) (see FIG. 6). In this analysis, the non-Alzheimer's group did not include Parkinson's disease patients.

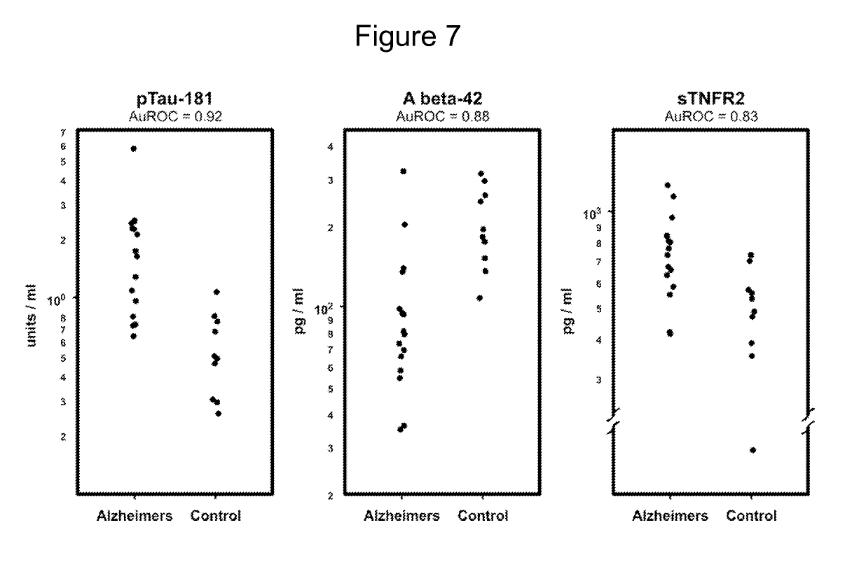

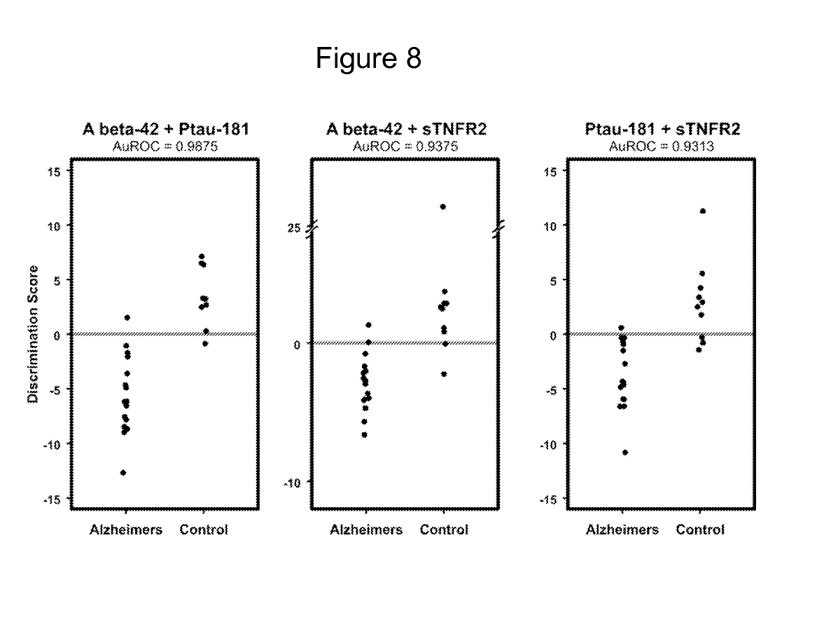

In order to provide a different means to visualize the difference between AD and controls (PD patients excluded) scatter plots for single biomarkers were generated. FIG. 7 demonstrates that even the known biomarkers Abeta-42 and pTau-181 may misclassify several subjects. Therefore scatter plots for two biomarkers that show the discrimination score from logistic regression models were generated. FIG. 8 demonstrates that the combinations of Abeta-42 and pTau-181, Abeta-42 and sTNFR2, or pTau-181 and sTNFR2 will misclassify less people than single biomarkers alone.

### Example 4

### Alzheimer's Disease Biomarkers Predict AD Severity

The mini-mental state examination (MMSE) or Folstein test is a brief 30-point questionnaire test that can be used to screen for cognitive impairment, and was used in this study to severity in AD patients. In order to determine which biomarkers best predict AD severity, an MMSE predictive algorithm was developed using the AD patients from the cohort and their MMSE correlation to subsets of biomarkers by linear combination of log-transformed biomarkers. The agreement between MMSE and biomarkers was assessed by Spearman rank correlation. Table 5 below includes a subset of biomarkers with the highest correlation with MMSE, and includes biomarkers with absolute Spearman correlation coefficients of 0.4. Surprisingly, IL-2 had the strongest correlation with MMSE and indicates higher IL-2 is associated with lower MMSE (Table 5). Biomarkers AB-oligomers and IL-22 also correlated well with MMSE. Additionally, biomarkers well correlated with MMSE, but poorly correlated with each other can for the basis for a good set of progression biomarkers (e.g., AB-oligomers and IL-2), and may represent independent pathways to progression prediction.

TABLE 5
Markers Most Correlated with MMSE (AD Patients Only)

| | | | | | | | | AB | |
|---|---|---|---|---|---|---|---|---|---|
| | MMSE | sTNFR2 | IL-2 | AB40 | GCSF | IL-22 | MMP2 | Oligomers | tMMP9 |
| MMSE | 1 | 0.43 | −0.71 | 0.46 | 0.48 | 0.61 | 0.49 | 0.6 | 0.46 |
| sTNFR2 | | 1 | −0.26 | 0.68 | 0.46 | 0.3 | 0.64 | 0.59 | 0.19 |
| IL-2 | | | 1 | −0.14 | −0.39 | −0.41 | −0.3 | −0.27 | −0.26 |
| AB40 | | | | 1 | 0.62 | 0.49 | 0.56 | 0.89 | 0.15 |
| GCSF | | | | | 1 | 0.52 | 0.5 | 0.55 | −0.04 |
| IL-22 | | | | | | 1 | 0.54 | 0.61 | 0.18 |
| MMP2 | | | | | | | 1 | 0.57 | −0.06 |
| AB Olig | | | | | | | | 1 | 0.31 |
| tMMP9 | | | | | | | | | 1 |

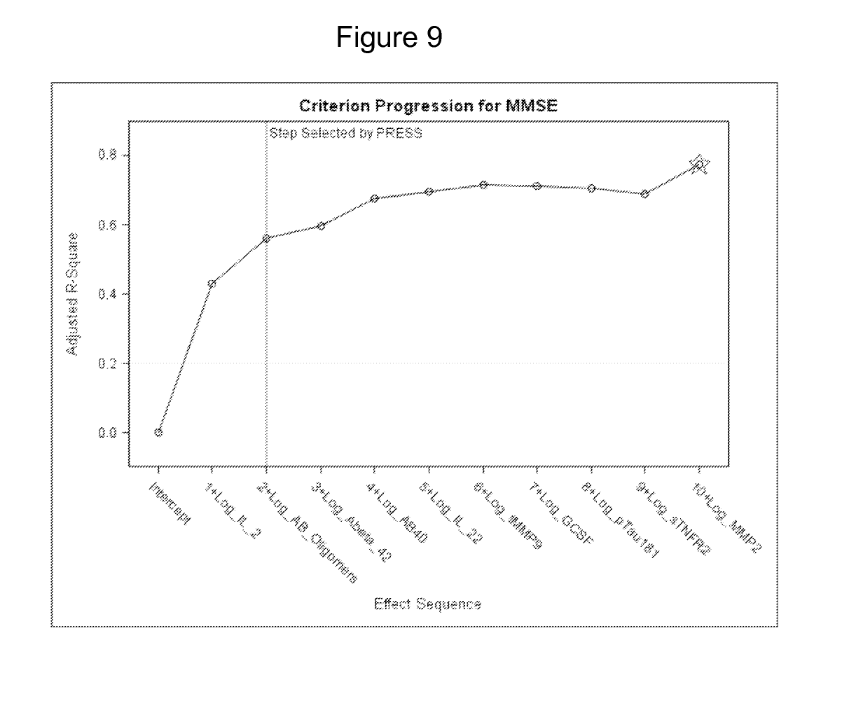

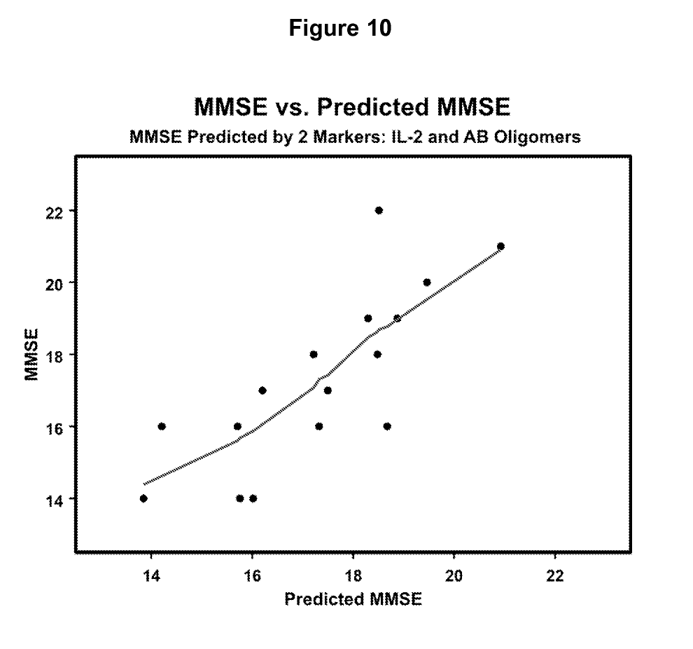

An algorithm was developed from a set of 8 biomarkers in Table 5 with r>0.4 (Abeta-42 and pTau-181 were also included); IL-2, AB-oligomers, Abeta-42, Abeta-40, IL-22, tMMP9, GCSF, pTau181, sTNFR2, and MMP2. A linear combination of biomarkers was generated to predict MMSE. General linear modeling techniques in SAS 9.2 were used, and agreement between the algorithm and MMSE was measured using correlation techniques (e.g., Spearman and Pearson). It was determined that the number of biomarkers in the algorithm is important and that adding more markers increases the correlation (see FIG. 9). However, too many markers may decrease the prediction of future datasets (robustness). Cross-validation was used to select the number of biomarkers, and FIG. 10 demonstrates the performance of a two-marker algorithm to predict MMSE using IL-2 and AB-oligomers. The Spearman rank correlation of r=0.78 was calculated using SAS 9.2 with a general linear model (4.10-3.13 log₁₀ (IL-2)+1.83 log₁₀ (AB-oligomers)) and biomarkers added by forward selection. The results demonstrate that MMSE score (a measure of AD severity) can be predicted with a combination of IL-2 and AB-oligomers (Abeta-oligomers); both of which exist at sub pg/mL concentrations in CSF.

All publications, patents, and patent applications mentioned in this specification are herein incorporated by reference to the same extent as if each individual publication, patent, or patent application was specifically and individually indicated to be incorporated by reference.

Although various specific embodiments of the present invention have been described herein, it is to be understood that the invention is not limited to those precise embodiments and that various changes or modifications can be affected therein by one skilled in the art without departing from the scope and spirit of the invention.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology, immunology, chemistry, biochemistry or in the relevant fields are intended to be within the scope of the appended claims.

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

1. A method of determining Alzheimer's Disease in a patient, comprising:
determining the concentrations of sTNFR2 and Abeta42 in a patient sample;
comparing the combined concentrations of sTNFR2 and Abeta42 in the sample to the combined concentrations of sTNFR2 and Abeta42 in a healthy population, and
determining Alzheimer's disease when the combined concentrations of sTNRF2 and Abeta42 in the patient sample is greater than the combined concentration in a healthy population.

2. The method of claim 1 wherein the sample comprises cerebral spinal fluid (CSF).

3. The method of claim 1, wherein the healthy population includes patients suffering from Parkinson's Disease.

4. The method of claim 1, wherein the healthy population does not include patients suffering from Parkinson's disease.

5. The method of claim 1, wherein the combined concentrations are determined using an area under curve analysis.

6. A method of determining Alzheimer's disease in a patient, comprising:
determining the concentrations of sTNFR2 and Ptau-181 in a patient sample;
comparing the combined concentration of sTNFR2 and Ptau-181 in the sample to a value reflecting the combined concentration of sTNFR2 and Ptau-181 in a healthy population, and
determining Alzheimer's disease when the combined concentration of sTNRF2 and Ptau-81 in the patient sample is greater than the combined concentration in a healthy population.

7. The method of claim 6 wherein the sample comprises cerebral spinal fluid.

8. The method of claim 6, wherein the healthy population includes patients suffering from Parkinson's Disease.

9. The method of claim 6, wherein the healthy population does not include patients suffering from Parkinson's disease.

10. The method of claim 6, wherein the combined concentration is determined using an area under curve analysis.

11. A method for determining Alzheimer's disease in a subject, comprising:
contacting, in vitro, a portion of a sample from the subject with a first antibody immunoreactive for Abeta42;
contacting, in vitro, a portion of the sample from the subject with a second antibody immunoreactive for sTNFR2;
contacting, in vitro, a portion of the sample from the subject with a third antibody immunoreactive for pTau-181;
determining the amounts of the Abeta42, sTNFR2 and pTau-181, and
determining the likelihood, presence or severity of Alzheimer's disease by comparing, in combination, the amounts of the Abeta42, sTNFR2 and pTau-181, with amounts, in combination, Abeta42, sTNFR2 and pTau-181, in a normal health population.

12. The method of claim 11, wherein the amount of Abeta42 in the normal healthy population is less than about 102 pg/ml.

13. The method of claim 11, wherein the amount of sTNFR2 in the healthy population is greater than about 748 pg/ml.

14. The method of claim 11, wherein the amount of Ptau-181 in the normal healthy population is greater than about 1.8 units/ml.

15-20. (canceled)
